(19) United States
(12) Patent Application Publication
(10) Pub. No.: US 2025/0149111 A1
(43) Pub. Date: May 8, 2025

(54) HYBRID PROTEIN DESIGN

(71) Applicants: Genentech, Inc., South San Francisco, CA (US); Hoffmann-La Roche Inc., Little Falls, NJ (US)

(72) Inventors: Simon Paul KELOW, Washington, DC (US); Daniel Joseph BERENBERG, New York, NY (US); Santrupti NERLI, South San Francisco, CA (US); Andrew Philip LEAVER-FAY, South San Francisco, CA (US); Jack Barton MAGUIRE, South San Francisco, CA (US); Michael Florian CHUNGYOUN, South San Francisco, CA (US); Andrew Martin WATKINS, San Francisco, CA (US); Jae Hyeon LEE, Boston, MA (US); Stephen Robert RA, New York, NY (US); Henri Vincent DWYER, South San Francisco, CA (US); Minji Maria LEE, Basel (CH); Kyunghyun CHO, New York, NY (US); Richard A. BONNEAU, New York, NY (US); Vladimir GLIGORIJEVIC, New Haven, CT (US)

(21) Appl. No.: 19/012,402

(22) Filed: Jan. 7, 2025

Related U.S. Application Data
(63) Continuation of application No. PCT/US2023/069796, filed on Jul. 7, 2023.
(60) Provisional application No. 63/359,167, filed on Jul. 7, 2022.

(51) Int. Cl. G16B 15/20; G16B 15/30; G16B 30/10

(52) U.S. Cl. CPC G16B 15/20; G16B 15/30; G16B 30/10

(57) ABSTRACT

A method may include applying a protein sequence computation model to generate, based on an input protein sequence, a plurality of proposed protein sequences. A set of possible amino acid residues for each position in at least a portion of an output protein sequence may be identified based on the plurality of proposed protein sequences. A first protein structure having the output protein sequence may be generated by applying a protein structure computation model to select, for each position in at least the portion of the output protein sequence, an amino acid residue from a corresponding set of possible amino acid residues. The protein structure computation model may further determine the conformation of the amino acid residues selected for inclusion in the output protein sequence. In some cases, the first protein structure may be grafted onto a second protein structure to form a third protein structure.

### CROSS REFERENCE TO RELATED APPLICATION

This application claims priority to U.S. Provisional Application No. 63/359,167, entitled “HYBRID PROTEIN DESIGN” and filed on Jul. 7, 2022, the disclosure of which is incorporated herein by reference in its entirety.

### TECHNICAL FIELD

The subject matter described herein relates generally to protein design and more specifically to protein design techniques that integrate protein sequence design with protein structure design.

### INTRODUCTION

Proteins are responsible for many essential cellular functions including, for example, enzymatic reactions, transport of molecules, regulation and execution of a number of biological pathways, cell growth, proliferation, nutrient uptake, morphology, motility, intercellular communication, and/or the like. A protein molecule may include one or more polypeptide chains, each of which including multiple amino acid residues linked together by peptide bonds. The primary structure of a molecule, dependent on the sequence of amino acid residues in the polypeptide chains forming the protein molecule, may refer to the protein molecule's three-dimensional structure (or conformation). For example, the secondary structure of the protein molecule may be defined at least in part by the torsion angles (or dihedral angles) of the peptide bonds present in the backbone (or main chain) of the protein molecule whereas the tertiary structure of the protein molecule may be defined by the folding of the polypeptide chains.

The functions of a protein molecule may be dependent on the sequence of amino acids in the polypeptide chains forming the protein molecule as well as the three-dimensional structure formed by the polypeptide chains. Accordingly, one key objective of protein design is to construct a novel sequence of amino acid residues (e.g., an antibody and/or the like) that exhibits certain desired properties, including the ability to adopt a certain three-dimensional structure. In the case of large molecule drug discovery, the novel protein sequence may be designed to fold into a three-dimensional structure that complements the three-dimensional structure of a target molecule (e.g., an antigen such as a viral antigen, a tumor antigen, and/or the like) and is sufficiently stable to allow the corresponding protein molecule to bind to the target molecule.

### SUMMARY

Systems, methods, and articles of manufacture, including computer program products, are provided for hybrid protein design. In some example embodiments, there is provided a system that includes at least one processor and at least one memory. The at least one memory may include program code that provides operations when executed by the at least one processor. The operations may include: identifying a protein sequence computation model and a protein structure computation model; applying the protein sequence computation model to generate, based at least on an input protein sequence, a plurality of proposed protein sequences; identifying, based at least on the plurality of proposed protein sequences, a set of possible amino acid residues for each position in at least a portion of an output protein sequence; and generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.

In another aspect, there is provided a method for hybrid protein design. The method may include: identifying a protein sequence computation model and a protein structure computation model; applying the protein sequence computation model to generate, based at least on an input protein sequence, a plurality of proposed protein sequences; identifying, based at least on the plurality of proposed protein sequences, a set of possible amino acid residues for each position in at least a portion of an output protein sequence; and generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.

In another aspect, there is provided a computer program product including a non-transitory computer readable medium storing instructions. The instructions may cause operations may executed by at least one data processor. The operations may include: identifying a protein sequence computation model and a protein structure computation model; applying the protein sequence computation model to generate, based at least on an input protein sequence, a plurality of proposed protein sequences; identifying, based at least on the plurality of proposed protein sequences, a set of possible amino acid residues for each position in at least a portion of an output protein sequence; and generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.

In some variations, one or more features disclosed herein including the following features can optionally be included in any feasible combination.

In some variations, the plurality of proposed protein sequences may be aligned to generate an aligned plurality of protein sequences. The set of possible amino acid residues for each position in at least the portion of the output protein sequence may be identified based at least on the aligned plurality of protein sequences.

In some variations, the plurality of proposed protein sequences may be aligned by applying one or more of dynamic programming, progressive alignment, hierarchical alignment, iterative alignment, motif finding, a deep learning model, and a Hidden Markov model.

In some variations, the identifying of the set of possible amino acid residues for each position in at least the portion of the output protein sequence may include identifying, for inclusion in the set of possible amino acid residues, a first amino acid residue but not a second amino acid residue.

In some variations, the identifying of the set of possible amino acid residues for each position in at least the portion of the output protein sequence may further include determining a first frequency at which a first amino acid residue appears at the position across the plurality of proposed protein sequences generated by the protein sequence computation model, determining a second frequency at which a second amino acid residue appears at the position across the plurality of proposed protein sequences generated by the protein sequence computation model, and identifying, based at least on the first frequency and the second frequency, the first amino acid residue but not the second amino acid residue for inclusion in the set of possible amino acid residues for the position.

In some variations, the first amino acid residue may be identified for inclusion in the set of amino acid residues based at least on the first frequency satisfying one or more thresholds. The second amino acid residue may be identified for exclusion from the set of possible amino acid residues based at least on the second frequency failing to satisfy the one or more thresholds.

In some variations, the identifying of the set of possible amino acid residues for the position in the output protein sequence may further include determining the one or more thresholds based on at least one of a maximum, a minimum, a median, a mean, and a mode of a frequency at which each of a plurality of amino acid residues appear at the position across the plurality of proposed protein sequences generated by the protein sequence computation model.

In some variations, the set of possible amino acid residues may include some but not all of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, and pyrrolysine.

In some variations, the protein structure computation model may generate the first protein structure by at least determining, based at least on an energy of the first protein structure having the output protein sequence, an identity and a conformation of an amino acid residue occupying each position in at least the portion of the output protein sequence.

In some variations, the protein structure computation model may determine the identity and the conformation of the amino acid residue occupying each position in at least the portion of the output protein sequence by at least modifying at least one of the identity and the conformation of the amino acid residue to minimize an energy of the first protein structure.

In some variations, the protein structure computation model may modify at least one of the identity and the conformation of the amino acid residue occupying a position by at least (i) changing a conformation of an amino acid residue occupying the position or (ii) selecting, from the set of amino acid residue associated with the position, a different possible amino acid residue for the position.

In some variations, the protein structure computation model may determine the identity and the conformation of the amino acid residue occupying each position in at least the portion of the output protein sequence by at least determining a first energy of the first protein structure having a first possible amino acid residue from the set of possible amino acid residues, determining a second energy of the first protein structure having a second possible amino acid residue from the set of possible amino acid residues, and generating the first protein structure to include, based at least on the first energy being lower than the second energy, the first possible amino acid residue instead of the second possible amino acid residue.

In some variations, the protein structure computation model may further determine the identity and the conformation of the amino acid residue occupying each position in at least the portion of the output protein sequence by at least determining a third energy of the first protein structure having a first conformation of the first possible amino acid residue, determining a fourth energy of the first protein structure having a second conformation of the first possible amino acid residue, and generating the first protein structure to include, based at least on the third energy being lower than the fourth energy, the first conformation of the first possible amino acid residue instead of the second conformation of the first possible amino acid residue.

In some variations, a property analysis model may be applied to determine a property of each protein sequence included in the plurality of proposed protein sequences. At least one protein sequence in the plurality of proposed protein sequences may be identified for exclusion based at least on the property of each protein sequence. The at least one protein sequence may be excluded from the plurality of proposed protein sequences prior to identifying, based at least on a remaining plurality of proposed protein sequences, the set of possible amino acid residues for each position in the output protein sequence.

In some variations, a first portion of a second protein structure may be identified. A third protein structure may be generated by at least replacing the first portion of the second protein structure with at least a second portion of the first protein structure.

In some variations, a third protein sequence of the third protein structure generated to include the second portion of the first protein structure and a third portion of the second protein structure may be determined. The protein structure computation model and/or a different protein structure computation model may be applied to determine, based at least on the third protein sequence, at least a fourth protein structure having the third protein sequence. A similarity metric quantifying a difference between the third protein structure and the fourth protein structure may be determined. The third protein sequence may be identified as a candidate for synthesis based at least on the similarity metric satisfying one or more thresholds.

In some variations, the second protein structure may be selected based at least on the second protein structure exhibiting one or more desired properties.

In some variations, the first portion of the second protein structure may include a first antigen binding site of a first antibody having the second protein structure the second portion of the first protein structure may include a second antigen binding site of a second antibody having the first protein structure.

In some variations, the first portion of the second protein structure may include a first paratope of a first antibody having the second protein structure and the second portion of the first protein structure may include a second paratope of a second antibody having the first protein structure.

In some variations, the first portion of the second protein structure may include a first complementarity determining region (CDR) of a first antibody having the second protein structure and the second portion of the first protein structure may include a second complementarity determining region (CDR) of a second antibody having the first protein structure.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to various protein design techniques that integrate protein sequence design with protein structure design, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a protein design system, in accordance with some example embodiments;

FIG. 2 depicts a flowchart illustrating an example of a process for hybrid protein design, in accordance with some example embodiments;

FIG. 3A depicts a flowchart illustrating another example of a process for hybrid protein design, in accordance with some example embodiments;

FIG. 3B depicts a flowchart illustrating another example of a process for hybrid protein design, in accordance with some example embodiments;

FIG. 4A depicts a flowchart illustrating another example of a process for hybrid protein design, in accordance with some example embodiments;

FIG. 4B depicts a schematic diagram illustrating an example of a process for verifying an in silico generated protein structure, in accordance with some example embodiments;

FIG. 5 depicts a flowchart illustrating another example of a process for hybrid protein design, in accordance with some example embodiments;

FIG. 6A depicts a schematic diagram illustrating an example of aligned protein sequences, in accordance with some example embodiments;

FIG. 6B depicts a schematic diagram illustrating an example of possible amino acid residue sets for different positions in a protein sequence, in accordance with some example embodiments;

FIG. 7A depicts a schematic diagram illustrating an example of a process for hybrid protein design in which the protein sequences output by a protein sequence computation model undergo structural design to generate a corresponding protein structure, in accordance with some example embodiments;

FIG. 7B depicts a schematic diagram illustrating another example of a process for verifying an in silico generated protein structure, in accordance with some example embodiments;

FIG. 8A depicts a schematic diagram illustrating an example of a process for hybrid protein design in which an alternate protein sequence is generated for the antibody trastuzumab, in accordance with some example embodiments;

FIG. 8B depicts graphs illustrating the human epidermal growth factor receptor 2 (HER2) binding affinity exhibited by the alternate protein sequences and structures for the antibody trastuzumab generated by various hybrid protein design workflows, in accordance withs some example embodiments;

FIG. 8C depicts a graph illustrating the antigen binding fragment (Fab) stability of the alternate protein sequences and structures generated for the antibody trastuzumab by various hybrid protein design workflows, in accordance withs some example embodiments; and

FIG. 9 depicts a block diagram illustrating an example of a computing system, in accordance with some example embodiments.

When practical, similar reference numbers denote similar structures, features, or elements.

### DETAILED DESCRIPTION

Protein design aims to identify novel protein sequences (e.g., sequences of amino acid residues) that exhibit certain desired properties including, for example, expression, binding affinity towards a target molecule (e.g., an antigen such as a viral antigen or a tumor antigen), specificity towards the target molecule, lack of nonspecificity, stability (e.g., conformation stability, thermodynamic stability, robustness to different environmental stresses such as protease resistance, and/or the like), non-immunogenicity, human-ness, absence of self-association (or non-aggregation), lack of chemical liabilities (e.g., aspartate isomerization, oxidation, deamidation), developability, and/or the like. However, protein design is a challenging and particularly resource intensive task at least because of the innumerable variations in protein sequence and structure. For example, the sequence space containing the possible permutation of amino acid residues that can form a protein molecule is vast (e.g., approximately 20^{N} for a protein sequence of N amino acid residues) but few of those possible permutations actually correspond to functional protein sequences. Meanwhile, the conformation space populated by the possible three-dimensional structures of a protein molecule is similarly immense even when confined to several discrete (instead of continuous) structural variations. For instance, a protein sequence of N amino acid residues has approximately 3^{N} possible conformations if each amino acid residue is limited to assuming one of three discrete geometric states (e.g., rotamers). As used herein, the term “space” denotes the solution space for a given problem. In the context of protein sequence design, the aforementioned sequence space may denote the possible solutions associated with constructing a proteins sequence (of specified or unspecified length) using a set of amino acid residues, such as the amino acid residues forming an antibody. In the case of protein structure design, the aforementioned conformation space may denote the possible solutions associated with determining the geometric state of amino acid residues forming a protein sequence including, for example, the geometric state of the backbone atoms and the sidechain atoms in each amino acid residue forming the protein sequence.

Accordingly, structurally agnostic prediction of protein sequences may yield many proteins that are unable to adopt three-dimensional structures that can bind with a target molecule. Predicting only protein structures may offer many possible protein structures that can structurally bind with a target antigen, but lack other desired properties (e.g., expression, specificity towards the target molecule, lack of nonspecificity, stability non-immunogenicity, human-ness, absence of self-association (or non-aggregation), lack of chemical liabilities, and/or the like). Both the sequence space and conformation space are so vast, many computationally predicted proteins may have little to no therapeutic value because the predicted protein sequences lack a structure that complements a target molecule and/or other desired properties. Thus, a segregated design approach that includes applying a protein structure computation model to an input protein sequence merely determines the three-dimensional structure adopted by the protein sequence but the protein sequence itself may remain suboptimal. For instance, a protein sequence generated separately (e.g., generated by a language based protein sequence computation model) may fail to exhibit certain desired properties, such as binding affinity and stability, at least because the language based protein sequence computation model lacks an awareness for the structural traits that contribute to these desired properties.

The present disclosure accounts for the suboptimal outcomes rendered by determining the sequence and structure of a protein molecule independently. The present systems and methods recognize that the three-dimensional structure and functions of a protein molecule are dependent on the sequence of amino acid residues forming the protein molecule (e.g., the primary structure of the protein molecule). For example, the binding affinity between a protein molecule and a target molecule may depend on whether the primary structure of the protein molecule is capable of adopting a three-dimensional structure (e.g., a secondary structure and a tertiary structure) that complements the three-dimensional structure of the target antigen. In some cases, the practical utility of the protein molecule as a therapeutic may further depend on the stability of its three-dimensional structure or portions thereof (e.g., the antigen binding fragment (Fab) and/or the like). Thus, determining a protein sequence that is more likely to exhibit certain desired properties may include a joint search across the sequence space and the conformation space to identify, for example, a permutation of amino acid residues capable of assuming a particular three-dimensional structure. As such, in some example embodiments, the present disclosure provides a protein design engine that may perform a hybrid protein design workflow that integrates protein sequence design and protein structure design, such that the generating of the sequence of amino acid residue forming a protein molecule is informed by the three-dimensional structure of the protein molecule. Nevertheless, a naïve hybrid protein design approach that combines a brute force search of the sequence space and conformation space is computationally intractable for protein sequences of meaningful length. Accordingly, as described in more detail below, the protein design engine may leverage the protein sequences generated by a protein sequence computation model, not as a standalone possible therapeutic proteins, but as sequences used to refine or guide the sequence space and the conformation space that are searched by the protein structure computation model when generating the sequence and the three-dimensional structure of a protein molecule. Doing so may reduce the computational burden associated with the hybrid protein design workflow while the resulting protein sequence may be more likely to exhibit one or more desired properties than those generated by a protein sequence model that operates without any structural awareness.

In some example embodiments, the protein design engine may perform a hybrid protein design workflow in which a protein structure computation model refines, based at least on the protein sequences generated by a protein sequence computation model, the sequence space and the conformation space that are searched to generate the sequence and the three-dimensional structure of a protein molecule. For example, in some cases, the protein design engine may apply the protein sequence computation model to generate, based at least on an input protein sequence, a plurality of proposed protein sequences. The protein sequence computation model may entail a language based protein sequence computation model and/or the like. In this context, a language based protein sequence computation model may refer to a machine learning model (or a deep learning model) that applies one or more natural language processing (NLP) techniques to generate protein sequences. Examples of machine learning architectures for the language based protein sequence computation model include autoencoders, transformers, long short term memory networks, recurrent neural networks, and/or the like.

In some cases, the input protein sequence may be selected as the basis for generating the plurality of proposed protein sequences at least because the input protein sequence exhibits one or more desired properties. The protein design engine may narrow the sequence space and the conformation space searched by the protein structure computation model by at least identifying, based at least on the plurality of proposed protein sequences, a set of possible amino acid residues for each position in at least a portion of an output protein sequence. As described in more detail below, the protein design engine may leverage the set of possible amino acid residues for each position in the output protein sequence to reduce the sequence space and the conformation space that are searched by the protein structure computation model by using various ways to exclude at least one possible amino acid residue at one or more positions in the output protein sequence. In doing so, the quantity of possible permutations of amino acid residues forming the output protein sequence as well as their corresponding conformations may be reduced.

In some cases, the protein design engine may reduce the sequence space and the conformation space searched by the protein structure computation model by applying a property analysis model. A property analysis model of the present disclosure may determine a property of each protein sequence in the plurality of proposed protein sequences generated by the protein sequence computation model. In some scenarios, least one protein sequence may be excluded from the plurality of proposed protein sequences based on its property. The quantity of possible amino acid residues for one or more positions in the output protein sequence may then be reduced by simply identifying the set of possible amino acid residues for each position in the output protein sequence based on the remaining plurality of proposed protein sequences. Doing so may further narrow the sequence space and the conformation space searched by the protein structure computation model.

Moreover, in some cases, the plurality of proposed protein sequences may be aligned to in order to identify the amino acid residues that appear at each position across the aligned plurality of proposed protein sequences. For instance, in some cases, the plurality of proposed protein sequences generated by the protein sequence computation model may be aligned by applying one or more aligning techniques such as dynamic programming, progressive alignment, hierarchical alignment, iterative alignment, motif finding, a deep learning model, a Hidden Markov model, and/or the like.

In some example embodiments, the protein design engine may determine, for each position in at least the portion of the output protein sequence, a set of possible amino acid residues that includes a first amino acid residue but not a second amino acid residue. For example, in some cases, the set of possible amino acid residues for a particular position in the output protein sequence may be identified to include the first amino acid residue based at least on a first frequency of the first amino acid residue appearing at the position across the plurality of proposed protein sequences generated by the protein sequence computation model. Alternatively and/or additionally, the set of possible amino acid residues for the position may be identified to exclude the second amino acid residue based at least on a second frequency of the second amino acid residue appearing at the position across the plurality of proposed protein sequences generated by the protein sequence computation model. Accordingly, in some cases, the set of possible amino acid residues for the position may be determined to include some but not all of the possible amino acid residues that can form a protein molecule. For instance, in cases where the output protein sequence corresponds to an antibody, the set of amino acid residues for the position may include some but not all of the amino acid residues that can form an antibody, which may include, for example, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and/or the like.

In some example embodiments, the protein design engine may generate a first protein structure having the output protein sequence by at least applying a protein structure computation model to select, for each position in at least the portion of the output protein sequence, an amino acid residue from the set of possible amino acid residues for inclusion in the output protein sequence. For example, in some cases, the protein structure computation model may generate the first protein structure by at least determining, based at least one or more criteria, a plurality of amino acid residues for inclusion in the output protein sequence and a corresponding conformation (or spatial arrangement) of the plurality of amino acid residues (e.g., the backbone and sidechain atoms of each amino acid residue). As noted, each position in the output protein sequence may be associated with a set of possible amino acid residues that contains, in some cases, a subset of every amino acid residue that can form an antibody. Accordingly, the selection of amino acid residues forming the output protein sequence may be limited to those included in each possible amino acid residue set. In some cases, the selection of amino acid residues forming the output protein sequence may be limited where one or more segments of the output protein sequence are designated for preservation. In this context, the preservation of a segment in the input protein sequence may entail preventing any insertion, deletions, or substitutions from affecting that segment when generating the output protein sequence. Thus, it should be appreciated that the identity of an amino acid residue in a preserved segment of the output protein sequence remains unchanged from that of the amino acid residue occupying the same position in the input protein sequence. That is, the same amino acid residues forming the preserved segment in the input protein sequence will remain present in the preserved segment in the output protein sequence. Accordingly, for a position in a preserved segment of the output protein sequence, the set of possible amino acid residues for that position may contain the single amino acid residue occupying the same position in the input protein sequence.

In some cases, the one or more criteria may include minimizing an energy function of the first protein structure having the plurality of amino acid residues selected for inclusion in the output protein sequence. Moreover, in some cases, the protein structure computation model may generate multiple protein molecules, each with a different sequence of amino acid residue and/or conformation before selecting the one protein molecule that satisfies the one or more criteria. The narrowing of the sequence space and the conformation space searched by the protein structure computation model may manifest in the drastic reduction in the quantity of possible protein molecules (e.g., having different combinations of amino acid residues and conformations) evaluated by the protein structure computation model. To further illustrate, the protein structure computation model may determine, for example, a first energy of a first protein molecule having a first plurality of amino acid residues and a first conformation. Furthermore, the protein structure computation model may generate a second protein molecule having a second plurality of amino acid residues and a second conformation by modifying at least one of the first plurality of amino acid residues and the first conformation. The protein structure computation model may determine a second energy of the second protein molecule. In instances where the first energy of the first protein molecule is lower than the second energy of the second protein molecule, the protein structure computation engine may generate the first protein structure to have the first plurality of amino acid residues and the first conformation (instead of the second plurality of amino acid residues and the second conformation). In some cases, the protein structure computation model may determine the first energy and the second energy by applying an energy function. For instance, the protein structure computation model may apply an energy function that is based on one or more of ab initio quantum mechanics, density functional theory (DFT), semiempirical methods, molecular mechanics force fields, statistical potentials, neural potentials, machine learning models (e.g., trained on structural data), and/or the like.

In some example embodiments, the protein structure computation model may further generate the first protein structure by at least determining a first backbone conformation of a first backbone of the first protein structure. The first backbone of the first protein structure may be a continuous chain of atoms formed by linking the backbone atoms of each amino acid residue in the output protein sequence. In some cases, the backbone atoms of each amino acid residue may be a sequence of atoms containing a nitrogen (N) atom, an α-carbon (c_{α}) atom, and a carboxyl carbon (C) atom. Accordingly, in some cases, the first backbone conformation of the first backbone may include the spatial arrangements of the backbone atoms in each amino acid residue selected for inclusion in the output protein sequence. Moreover, in some cases, the spatial arrangements of the backbone atoms may be defined by one or more of a translation of the first backbone, a rotation of the first backbone, and/or a torsion angle of one or more rotatable bonds formed by the backbone atoms in the first backbone (e.g., the torsion angle ψ of the rotatable bond between the a-carbon (c_{α}) atom and the carbonyl group, the torsion angle ϕ of the rotatable bond between the a-carbon (c_{α}) atom and the nitrogen (N) atom, the torsion angle ω of the rotatable bond between the carbon (C) atom and the nitrogen (N) atom, and/or the like).

In some example embodiments, the protein structure computation model may determine the first backbone conformation of the first backbone of the first protein structure to have a same conformation as at least a portion of a second backbone of a second protein structure. In some cases, the second protein structure may be associated with a protein sequence having one or more desired properties such as expression, binding affinity towards a target molecule, specificity towards the target molecule, lack of nonspecificity, stability, non-immunogenicity, human-ness, absence of self-association (or non-aggregation), and/or the like. For example, in some cases, that protein sequence may be the input protein sequence based on which the protein sequence computation model generated the plurality of proposed protein sequences or a third protein sequence. Furthermore, in some cases, the second backbone of the second protein structure may exhibit a second backbone conformation of the second protein structure in an unbound state or a third backbone conformation of the second protein structure bound to a target molecule.

In some example embodiments, instead of the first backbone conformation of the first backbone being determined to have a same conformation as at least the portion of the second backbone of the second protein structure, the protein structure computation model may determine the first backbone conformation of the first backbone by at least determining the geometric state of the first backbone. In some cases, the geometric state of the first backbone may be defined by the translation and/or rotation of the backbone atoms included in each amino acid residue in the output protein sequence such that the protein structure computation model determines the geometric state of the first backbone by at least determining the translation and/or rotation of the backbone atoms. Alternatively and/or additionally, the geometric state of the first backbone may be defined by the torsion angle of one or more rotatable bonds formed by the backbone atoms in each amino acid residue in the output protein sequence. Accordingly, in some cases, the protein structure computation model may determine the geometric state of the first backbone by at least determining the torsion angle of the one or more rotatable bonds formed by the backbone atoms.

In some example embodiments, upon determining the first backbone conformation of the first backbone of the first protein structure, the protein design computation model may further generate the first protein structure by at least determining to include, at each position in at least a portion of the first backbone having the first backbone conformation, the amino acid residue selected for inclusion at a corresponding position in the output protein sequence. Furthermore, the protein structure computation model may generate the first protein structure by at least determining, for each position in at least a portion of the first backbone having the first backbone conformation, a sidechain conformation of the amino acid residue selected for inclusion at a corresponding position in the output protein sequence. For example, in some cases, the sidechain conformation of the amino acid residue may be determined based at least on the first backbone conformation of the first backbone of the first protein structure. Moreover, the protein structure computation model may determine the sidechain conformation of the amino acid residue selected for inclusion in the output protein sequence by at least determining a geometric state of the sidechain. For instance, in some cases, the geometric state of the sidechain of the amino acid residue may be determined by at last selecting a rotamer from a plurality of possible rotamers, each of which including a different combination of torsion angles of the rotatable bonds formed by the sidechain atoms in the amino acid residue, Alternatively, the geometric state of the sidechain may be determined by determining one or more of a translation, a rotation, and/or a torsion angle of the sidechain atoms in the amino acid residue.

In some example embodiments, the protein design engine may verify the first protein structure generated by the protein structure computation model by at least applying a different protein structure computation model to determine, based at least on the output protein sequence, at least a second protein structure having the output protein sequence. Furthermore, the protein design engine may verify the first protein structure generated by the protein structure computation model by determining a similarity metric (e.g., a root mean square deviation (RMSD) and/or the like) quantifying the difference between the first protein structure and the second protein structure. In cases where the similarity metric satisfies one or more thresholds, the protein design engine may verify the first protein structure and identify the corresponding output protein sequence as a candidate for synthesis, in vitro measurements, in vivo characterizations, and/or the like.

In some example embodiments, the protein design engine may generate the first protein structure such that one or more portions of the first protein structure may be used as donor structures for grating onto corresponding portions of a second protein structure. For example, in some cases, the second protein structure may be selected at least because the second protein structure exhibits one or more desired properties such as binding affinity towards a target molecule, specificity towards the target molecule, lack of nonspecificity, stability, non-immunogenicity, human-ness, absence of self-association (or non-aggregation), and/or the like. Accordingly, in some cases, the protein design engine may identify a first portion of the second protein structure before generating a third protein structure by at least replacing the first portion of the second protein structure with a second portion of the first protein structure. In some cases, the protein design engine may verify the third protein structure based at least on a similarity metric (e.g., root mean square deviation (RMSD) and/or the like) quantifying a difference between the third protein structure and at least a fourth protein structure generated by the protein structure computation model and/or a different protein structure computation model based on a third protein sequence of the third protein structure. In instances where the first protein structure and the second protein structure are antibodies, the first portion of the second protein structure may include a first antigen binding site (e.g., a first paratope, a first complementarity determining region (CDR), and/or the like) while the second portion of the first protein structure may include a second antigen binding site (e.g., a second paratope, a second complementarity determining region (CDR), and/or the like). Grafting the second portion of the first protein structure onto the second protein structure may therefore generate an antibody whose sequence and structure combine the sequences and structures of multiple antibodies.

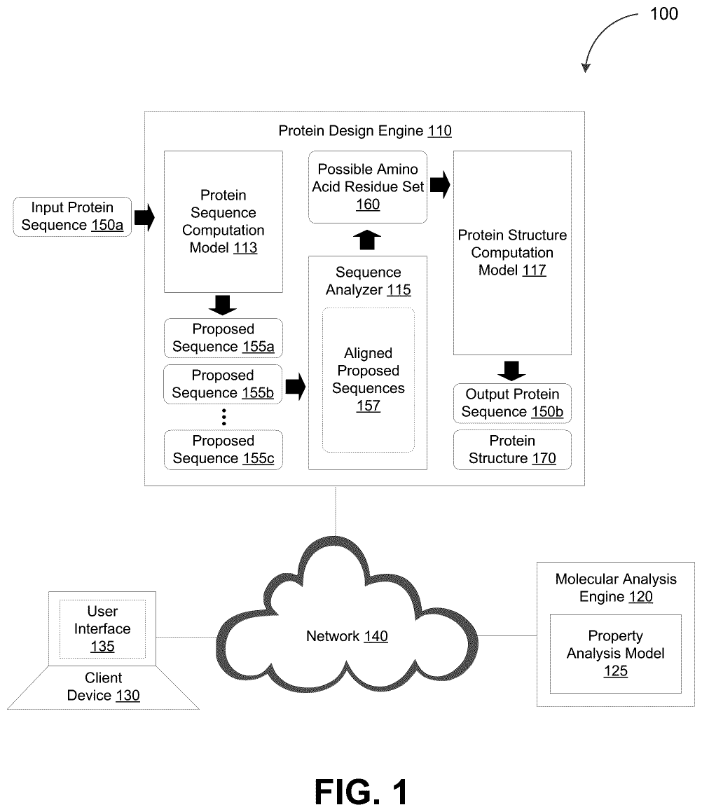

FIG. 1 depicts a system diagram illustrating an example of a protein design system 100, in accordance with some example embodiments. Referring to FIG. 1, the protein design system 100 may include a protein design engine 110, a molecular analysis engine 120, and a client device 130. As shown in FIG. 1, the protein design engine 110, the analysis engine 120, and the client device 130 may be communicatively coupled via a network 140. The client device 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the protein design engine 110 may apply a protein sequence computation model 113 to generate, based on an input protein sequence 150a, a plurality of proposed protein sequences 155 including, for example, a first proposed protein sequence 155a, a second proposed protein sequence 155b, a third proposed protein sequence 155c, and/or the like. In some cases, the protein design engine 110 may receive the input protein sequence 150a, which includes a sequence of amino acid residues, from the client device 130. Moreover, in some cases, the input protein sequence 150a may be selected as the basis for generating the plurality of proposed protein sequences 155 at least because the input protein sequence 150a exhibits one or more desired properties including, for example, expression, binding affinity towards a target molecule (e.g., an antigen such as a viral antigen or a tumor antigen), specificity towards the target molecule, lack of nonspecificity, stability, non-immunogenicity, human-ness, absence of self-association (or non-aggregation), and/or the like.

In some example embodiments, the protein sequence computation model 113 may include one or more machine learning models trained to generate the plurality of proposed protein sequences 155. For example, in some cases, the one or more machine learning models may generate each protein sequence of the plurality of proposed protein sequences 155 by at least sampling, based on the input protein sequence 150a, a data distribution learned by the one or more machine learning models during training. That is, each sampling of the data distribution may correspond to a single sampling iteration that generates a single protein sequence of the plurality of proposed protein sequences 155. For instance, a first sampling of the data distribution may generate the first proposed protein sequence 155a while a second sampling of the data distribution may generate the second proposed protein sequence 155b. In some cases, the one or more machine learning models may be trained based on a variety of known protein sequences, including protein sequences known to exhibit certain functions as well as protein sequences without any known functions. Accordingly, the one or more machine learning models may be trained to learn a data distribution corresponding to a reduced dimension representation of the sequences of amino acid residues forming the known protein sequences.

In some cases, the one or more machine learning models may include an autoencoder (e.g., a denoising autoencoder (DAE) and/or the like), in which case the one or more machine learning models may learn the data distribution by at least learning to generate an encoding of an input protein sequence that is then decoded to form an output protein sequence that is minimally different from the input protein sequence. At inference time, the data distribution associated with the trained autoencoder may be sampled by encoding, for example, the input protein sequence 150a before decoding an intermediate sequence having at least one of a corruption (e.g., an insertion, a deletion, and/or a modification of an amino acid residue) and a length change relative to the input protein sequence 150a. Moreover, the sampling of the data distribution may be guided by the properties of the intermediate sequences. For example, in some cases, an intermediate sequence sampled from the data distribution may undergo property analysis (e.g., by a computational function prediction model) and may be decoded if the intermediate sequence is determined to exhibit one or more desired properties, which may be also present in the input protein sequence 150a. In cases where the intermediate sequence fails to exhibit the one or more desired properties, another intermediate sequence may be generated by sampling the data distribution before that intermediate sequence is also subjected to property analysis. Accordingly, the intermediate sequence that is decoded to generate each of the plurality of proposed protein sequences 155 may be different than the input protein sequence 150a but will still exhibit the same (or similar) desired properties as the input protein sequence 150a. For instance, the first proposed protein sequence 155a, the second proposed protein sequence 155b, and the third proposed protein sequence 155c may be different from the input protein sequence 150a but will retain at least some of the desired properties of the input protein sequence 150a.

In some example embodiments, the protein design engine 110 may apply a variety of sampling techniques to sample from the data distribution including, for example, a Markov Chain Monte Carlo (MCMC), importance sampling (IS), rejection sampling, Metropolis-Hastings, Gibbs sampling, slice sampling, exact sampling, and/or the like. Moreover, as noted, each sampling of the data distribution may correspond to a single sampling iteration generating one proposed protein sequence of the plurality of proposed protein sequences 155. In some cases, the protein design engine 110 may continue to apply the protein sequence computation model 113 to sample the data distribution until one or more conditions are satisfied. For example, in some cases, the protein design engine 110 may continue to apply the protein sequence computation model 113 to sample the data distribution until the plurality of proposed protein sequences 155 generated by the protein sequence computation model 113 includes a threshold quantity of proposed protein sequences. Alternatively and/or additionally, each proposed protein sequence of the plurality of proposed protein sequences 155 generated by the protein sequence computation model 113 may undergo molecular analysis (e.g., by the molecular analysis engine 120 applying a property analysis model 125) in order to determine one or more properties of each proposed protein sequence. In those instances, the protein design engine 110 may continue to apply the protein sequence computation model 113 to generate additional proposed protein sequences until the plurality of proposed protein sequences 155 generated by the protein sequence computation model 113 includes a threshold quantity of proposed protein sequences whose properties satisfy one or more thresholds. Accordingly, in some cases, the plurality of proposed protein sequences 155 may exclude one or more proposed protein sequences whose properties (e.g., as determined by the property analysis model 125) fails to satisfy the one or more thresholds.

In some example embodiments, the protein design engine 110 may perform variable-length sampling in which the plurality of proposed protein sequences 155 that undergo further analysis have different lengths or quantity of constituent amino acid residues. For example, in some cases, the first proposed sequence 155a may have a different length (or be formed from a different quantity of amino acid residues) than the second proposed sequence 155b and/or the third proposed sequence 155c. Alternatively, in some cases, the protein design engine 110 may perform fixed-length sampling in which the plurality of proposed protein sequences 155, including the first proposed sequence 155a, the second proposed sequence 155b, and the third proposed sequence 155c, and/or the like, have a same length (or are formed from a same quantity of amino acid residues). In some cases, the protein design engine 110 may implement fixed-length sampling by at least training the protein sequence computation model 113 based on a training dataset that contains protein sequences of the same length. In doing so, the data distribution that is learned by the protein sequence computation model 113 may be populated by encodings of protein sequences that decode to the same-length protein sequence. Accordingly, the plurality of proposed protein sequences 155 that are generated by applying the protein sequence computation model 113 may also have a same length (or same quantity of constituent amino acid residues). In cases where the protein sequence computation model 113 is trained based on different length protein sequences, the protein sequence computation model 113 may generate protein sequences that have different lengths (or different quantities of constituent amino acid residues). In those instances, the protein design engine 110 may implement fixed-length sampling by at least excluding, from the plurality of proposed protein sequences 155, any protein sequence generated by the protein sequence computation model 113 that do not have a particular length (or particular quantity of constituent amino acid residues).

Referring again to FIG. 1, in some example embodiments, the protein design engine 110 may include a sequence analyzer 115 that determines, based at least on the plurality of proposed protein sequences 155 generated by the protein sequence computation model 113, a possible amino acid residue set 160 for each position in an output protein sequence 150b. As noted, in cases where the protein design engine 110 performs fixed-length sampling, the plurality of proposed protein sequences 155 may include protein sequences having a same length (or same quantity of constituent amino acid residues) whereas in the case of variable-length sampling, the plurality of proposed protein sequences 155 may be different in length (or quantity of constituent amino acid residues). In some cases, the sequence analyzer 115 may align the plurality of proposed protein sequences 155 in order to identify the amino acid residues that appear at each position across an aligned plurality of proposed protein sequences 157. For example, in some cases, the sequence analyzer 115 may generate the aligned plurality of proposed protein sequences 157 by at least applying, to the plurality of proposed protein sequences 155, one or more of dynamic programming, progressive alignment, hierarchical alignment, iterative alignment, motif finding, a deep learning model, a Hidden Markov model, and/or the like. As described in more details below, the possible amino acid residue set 160 for at least some of the positions in the output protein sequence 150b may include a subset of amino acid residues containing some but not all of the possible constituent amino acid residues of a protein molecule. In instances where the output protein sequence 150b corresponds to an antibody, the possible amino acid residue set 160 may include some but not all of the amino acid residues that can form an antibody such as, for example, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and/or the like.

In some example embodiments, the protein design engine 110 may apply a protein structure computation model 117 to generate a first protein structure 170 by at least selecting, for each position in at least a portion of the output protein sequence 150b, an amino acid residue from the corresponding possible amino acid residue set 160 for inclusion in the output protein sequence 150b. In some cases, the protein structure computation model 117 may include a physics-based protein structure computation model, a machine learning based protein structure computation model, and/or the like. As will be described in more details below, the protein structure computation model 117 may generate the first protein structure 170 by at least determining a first backbone conformation of a first backbone of the first protein structure 170. Furthermore, the protein structure computation model 117 may generate the first protein structure 170 by at least determining, for each position in at least a portion of the first backbone having the first backbone conformation, a sidechain conformation of the amino acid residue selected for inclusion at a corresponding position in the output protein sequence 150b.

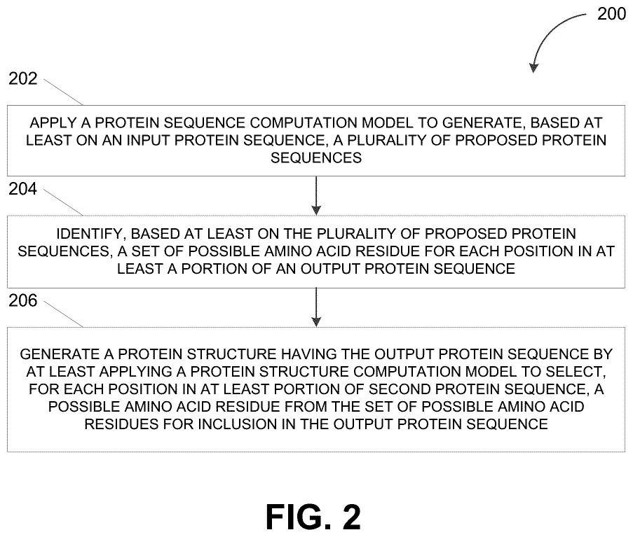

FIG. 2 depicts a flowchart illustrating an example of a process 900 for hybrid protein design, in accordance with some example embodiments. Referring to FIGS. 1 and 2, the process 900 may be performed by the protein design engine 110 to generate, based at least on the input protein sequence 150a, the output protein sequence 150b and the corresponding first protein structure 170.

At 202, the protein design engine 110 may apply the protein sequence computation model 113 to generate, based at least on an input protein sequence, a plurality of proposed protein sequences. In some example embodiments, the protein sequence computation model 113 may generate, based on the input protein sequence 150a, the plurality of proposed protein sequences 155 including, for example, the first proposed protein sequence 155a, the second proposed protein sequence 155b, the third proposed protein sequence 155c, and/or the like. In some cases, the protein sequence computation model 113 may generate the plurality of proposed protein sequences 155 by at least sampling a data distribution populated by encodings of various protein sequences. The data distribution may be learned by the protein sequence computation model 113 by at least training the protein sequence computation model 113 to encode known protein sequences, including protein sequences known to exhibit certain functions and protein sequences without any known functions, such that the known protein sequences may be recovered (e.g., through decoding) from the corresponding encodings with minimal information loss (e.g., difference between the decoded protein sequences and the original protein sequences). With each sampling of the data distribution, the protein sequence computation model 113 may generate a single proposed protein sequence that differs from the input protein sequence 150a, for example, by having at least one of a corruption (e.g., an insertion, a deletion, and/or a modification of an amino acid residue) and a length change relative to the input protein sequence 150a. As noted, the sampling of the data distribution may be guided by the properties of the intermediate sequences such that the plurality of proposed sequences 155 generated therefrom may exhibit certain desired properties including, in some cases, the same (or similar) properties as the input protein sequence 150a.

At 204, the protein design engine 110 may identify, based at least on the plurality of proposed protein sequences, a set of possible amino acid residues for each position in at least a portion of an output protein sequence. In some example embodiments, the sequence analyzer 115 may determine, based at least on the plurality of proposed protein sequences 155, the possible amino acid residue set 160 for each position in the output protein sequence 150b. As shown in FIG. 1, in some cases, the sequence analyzer 115 may align the plurality of proposed protein sequence 155, for example, by applying one or more aligning techniques including, for example, dynamic programming, progressive alignment, hierarchical alignment, iterative alignment, motif finding, a deep learning model, a Hidden Markov model, and/or the like. In doing so, the sequence analyzer 115 may generate the aligned proposed sequences 157 before determining, based at least on the aligned proposed sequences 157, the possible amino acid residue set 160 for each position in the output protein sequence 150b.

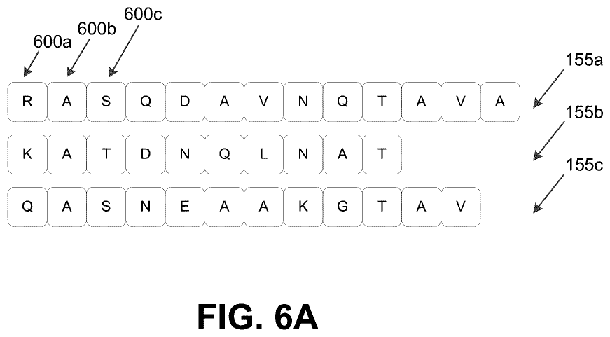

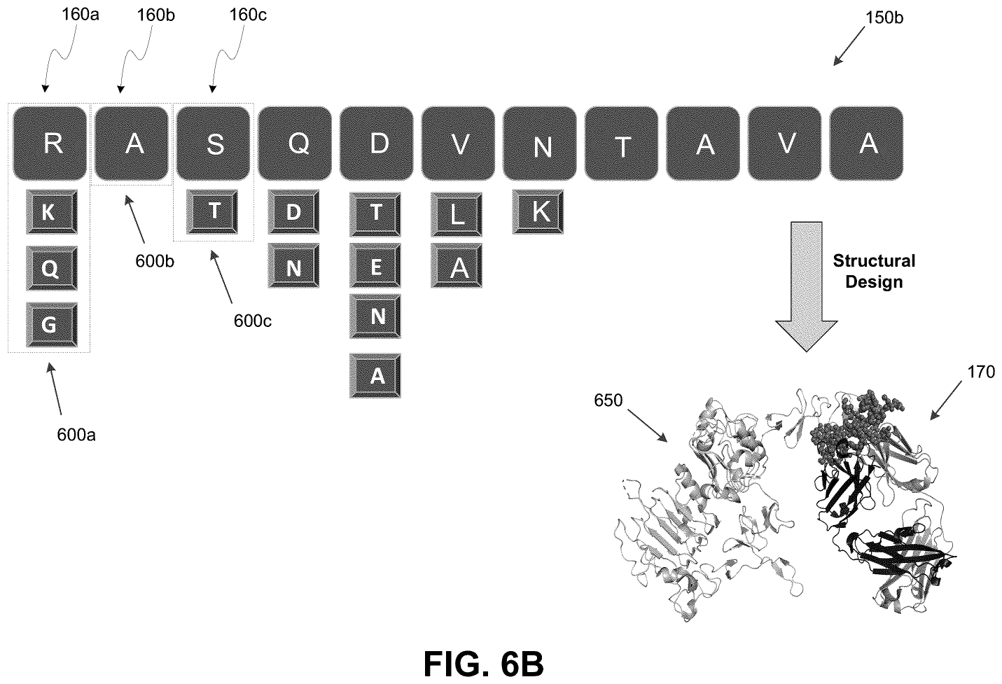

To further illustrate, FIG. 6A depicts a schematic diagram illustrating an example of the aligned proposed sequences 157 generated by the sequence analyzer 115 aligning at least the first proposed sequence 155a, the second proposed sequence 155b, and the third proposed sequence 155c. In some example embodiments, once the plurality of proposed protein sequences 155 are aligned, the sequence analyzer 115 may identify the amino acid residues that appear at each position across the aligned proposed sequences 157. Moreover, in some cases, the sequence analyzer 115 may determine the frequency with which each amino acid residue appears at each position across the aligned proposed sequences 157. In the example shown in FIG. 6A, once the first proposed sequence 155a, the second proposed sequence 155b, and the third proposed sequence 155c are aligned, the sequence analyzer 115 may determine that the amino acid residue arginine (R) appears in a first position 600a of the first proposed sequence 155a while the amino acid residue lysine (K) appears at the first position 600a of the second proposed sequence 155b and the amino acid residue glutamine (Q) appears in the first position 600a of the third proposed sequence 155c. The sequence analyzer 115 may also determine that the amino acid residue alanine (A) appears in a second position 600b of the first proposed sequence 155a, the second proposed sequence 155b, and the third proposed sequence 155c. Furthermore, the sequence analyzer 115 may determine that the amino acid residue serine(S) appears in a third position 600c of the first proposed sequence 155a and the third proposed sequence 155c while the amino acid residue threonine (T) appears in the third position 600c of the second proposed sequence 155b.

In some example embodiments, the sequence analyzer 115 may determine, based at least on the aligned proposed sequences 157, the amino acid residues for which the respective likelihood of appearing in the output protein sequence 150b satisfies one or more thresholds. For example, in some cases, the sequence analyzer 115 may identify, based at least on the amino acid residues occupying each position across the aligned proposed sequences 157, the amino acid residues that are most likely to occupy each position in at least a portion of the output protein sequence 150b. Accordingly, in some cases, the sequence analyzer 115 may generate the possible amino acid residue set 160 of each position to exclude at least some of the amino acid residues that can form a protein molecule. Doing so may reduce the sequence space of possible permutations of amino acid residues from the protein structure computation model 117 determines the output protein sequence 150b and the corresponding protein structure 170 during subsequent structural design.

In some example embodiments, the sequence analyzer 115 may determine, based at least on the amino acid residues that appear in each position across the aligned proposed sequences 157, the possible amino acid residue set 160 for each position in at least a portion of the output protein sequence 150b. For example, the possible amino acid residue set 160 for a particular position may be identified to include amino acid residues that are more likely to occupy the position in the output protein sequence 150b and exclude amino acid residues that are less likely to occupy the position in the output protein sequence 150b. In instances where the output protein sequence 150b is an antibody, the possible amino acid residue set 160 for the position may include a subset of the amino acid residues that can form an antibody. For instance, where the output protein sequence 150b is an antibody, the sequence analyzer 115 may determine, based at least on the amino acid residues that appear in each position across the aligned proposed sequence 157, some but not all of the amino acid residues alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In some cases, one or more amino acid residues may be identified, based at least on the frequency with which each amino acid residue appears at the position, for inclusion in the possible amino acid residue set 160 of a particular position in the output protein sequence 150b. For example, a first amino acid residue may be identified for inclusion in the possible amino acid residue set 160 of a position if the frequency with which the first amino acid residue appears in that position across the aligned proposed sequences 157 indicates that the first amino acid residue is likely to occupy that position in the output protein sequence 150b. Conversely, a second amino acid residue may be excluded from the possible amino acid residue set of the position if the frequency with which the second amino acid residue appears in that position across the aligned proposed sequences 157 indicates that the second amino acid residue is unlikely to occupy that position in the output protein sequence 150b. Accordingly, in some cases, the one or more amino acid residues may be identified for inclusion in the possible amino acid residue set 160 for a position in the output protein sequence 150b if the frequency with which the one or more amino acid residues appear at that position across the aligned proposed sequences 157 satisfies one or more thresholds. In some cases, the one or more thresholds may be determined based on a maximum, a minimum, a median, a mean, and a mode of a frequency at which each of a plurality of amino acid residues appear at the position across the aligned proposed sequences 157. For instance, in some cases, a first amino acid residue may be identified for inclusion in the possible amino acid residue set 160 of a particular position in the output protein sequence 150b if a first frequency with which the first amino acid residue appears in that position across the aligned proposed sequences 157 exceed a median frequency at which each of a plurality of amino acid residues appear at the position across the aligned proposed sequences 157. Contrastingly, a second amino acid residue may be excluded from the possible amino acid residue set 160 of the position if a second frequency with which the second amino acid residue appears in the position across the aligned proposed sequences 157 fails to exceed the aforementioned median frequency.

To further illustrate, FIG. 6B depicts a schematic diagram illustrating an example of the possible amino acid residue set 160 that is generated for each position in at least a portion of the output protein sequence 150b. In the example shown in FIG. 6B, the first position 600a of the output protein sequence 150b may be associated with a first possible amino acid residue set 160a that includes the amino acid residues arginine (R), lysine (K), glycine (Q), and glutamine (Q). The sequence analyzer 115 may select the amino acid residues arginine (R), lysine (K), glycine (Q), and glutamine (Q) for inclusion in the first possible amino acid residue set 160a at least because the frequency with which these amino acid residues appear in the first position 160a across the aligned proposed sequence 157 satisfies one or more thresholds. Meanwhile, the second position 600b of the output protein sequence 150b may be associated with a second possible amino acid residue set 160b that includes the amino acid residue alanine (A), which may be selected for inclusion based at least on the frequency with which the amino acid residue alanine (A) appears in the second position 600b across the aligned proposed sequences 157 satisfying one or more thresholds. Furthermore, the third position 600c of the output protein sequence 150b may be associated with a third possible amino acid residue set 160c that includes the amino acid residues serine(S) and threonine (T). The amino acid residues serine(S) and threonine (T) may be included in the third possible amino acid residue set 160c of the third position 600c at least because the frequency at which these amino acid residues appear in the third position 600c across the aligned proposed sequences 157 satisfies one or more thresholds. It should be appreciated that the amino acid residues that are absent from each possible amino acid residue set 160 may be excluded at least because the frequency with which these amino acid residues appear at the corresponding position across the aligned proposed sequences 157 fails to satisfy one or more thresholds.

At 206, the protein design engine 110 may generate a protein structure having the output protein sequence by at least applying the protein structure computation model 117 to select, for each position in at least a portion of the output protein sequence, an amino acid residue from a corresponding set of possible amino acid residues for inclusion in the output protein sequence. In some example embodiments, the protein design engine 110 may apply the protein structure computation model 117, which may generate the first protein structure 170 having the output protein sequence 150b by at least selecting, for each position in at least a portion of the output protein sequence 150b, an amino acid residue from the corresponding possible amino acid residue set 160 for inclusion in the output protein sequence 150b. That the protein structure computation model 117 operates on the possible amino acid residue set 160 for each position, which contains some but not all of the possible amino acid residues forming a protein molecule, reduces the computational complexity of the structural design performed by the protein structure computation model 117. For example, the protein structure computation model 117 may generate the first protein structure 170 having the output protein sequence 150b by at least evaluating various possible protein molecules, each of which having a different permutation of amino acid residues and conformation. Accordingly, to generate the first protein structure 170 having the output protein sequence 150b, the protein structure computation model 117 may perform a joint search across the sequence space of possible permutations of amino acid residues forming a protein molecule and the conformation space of the corresponding three-dimensional structure. The computational burden of this search may be reduced by the protein structure computation model 117 leveraging the possible amino acid residue set 160 of each position to drastically reduce the quantity of possible protein molecules that are evaluated in order to generate the output protein sequence 150b.

In some cases, the protein structure computation model 117 may perform the joint search across the sequence space and the conformation space by at least evaluating the three-dimensional structure of different permutations of amino acid residues formed by the possible amino acid residue in each set of possible amino acid residue 160. For instance, in some cases, the protein structure computation model 117 may apply determine the energy of each three-dimensional protein structure in order to identify a permutation of amino acid residues that folds into a three-dimensional structure having a minimal energy. As described in more details below, for each permutation of amino acid residues forming the output protein sequence 150b, the protein structure computation model 117 may determine the corresponding first protein structure 170 by at least determining the backbone conformation of the first protein structure 170 having that particular permutation of amino acid residues and/or the sidechain conformation of the constituent amino acid residues. For example, in some cases, the protein structure computation model 117 may determine the geometric states of the backbone of the first protein structure 170 and/or sidechains of the constituent amino acid residues with varying degrees of freedom. In the case of backbone conformation, the protein structure computation model 117 may determine the geometric state of the backbone of the first protein structure 170 based at least partially on the backbone conformation of another protein structure associated with one or more desired properties (e.g., expression, binding affinity towards a target molecule (e.g., an antigen such as a viral antigen or a tumor antigen), specificity towards the target molecule, lack of nonspecificity, stability, non-immunogenicity, human-ness, absence of self-association (or non-aggregation), and/or the like). Meanwhile, the sidechain conformation of an amino acid residue may be determined by selecting one of a plurality of possible rotamers or by determining the torsion angle of one or more rotatable angle formed by the sidechain atoms of the amino acid residue. FIG. 6B shows one example of structural design in which the first protein structure 170 generated by the protein structure computation model 117 is in a bound state in which the first protein structure 170 is bound to a target molecule 650. However, it should be appreciated that the protein structure computation model 117 may generate the first protein structure 170 to be in a bound state or an unbound state.

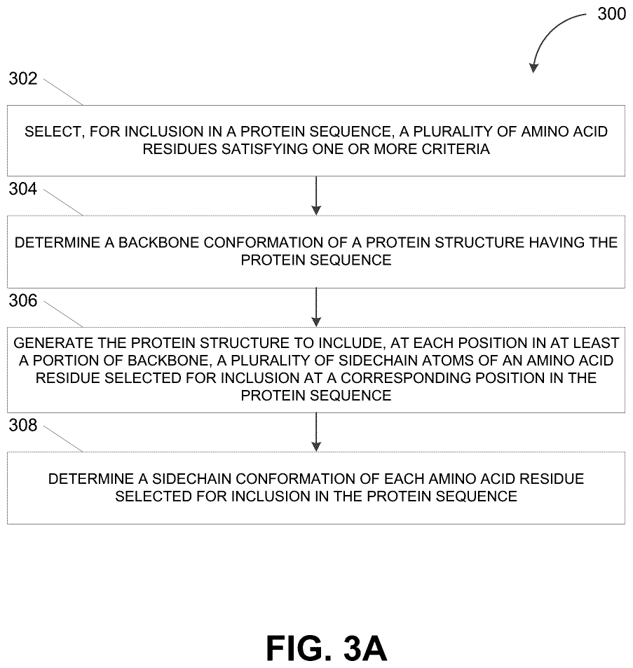

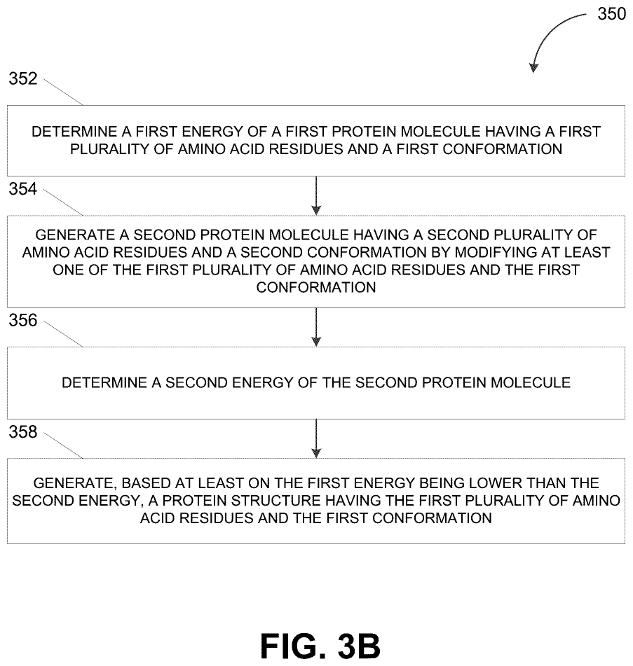

FIG. 3A depicts a flowchart illustrating an example of a process 300 for hybrid protein design, in accordance with some example embodiments. Referring to FIGS. 1-2 and 3A, the process 300 may be performed by the protein design engine 110 to generate, based at least on the input protein sequence 150a, the output protein sequence 150b and the corresponding first protein structure 170. In some cases, the process 300 may implement operation 206 of the process 200 in which the protein structure computation model 117 selects the constituent amino acid residues of a protein sequence and determines the corresponding protein structure by at least determining the conformation of the backbone and sidechains of the protein structure.

At 302, the protein structure computation model 117 may select, for inclusion in a protein sequence, a plurality of amino acid residues satisfying one or more criteria. In some example embodiments, the protein design engine 110 may apply the protein structure computation model 117 to select, for each position in at least a portion of the output protein sequence 150b, an amino acid residue from the corresponding possible amino acid residue set 160 for inclusion in the output protein sequence 150b. Referring again to the example shown in FIG. 6B, the protein structure computation model 117 may be applied to select, for example, the amino acid residue arginine (R) from the first possible amino acid residue set 160a, the amino acid residue alanine (A) from the second possible amino acid residue set 160b, and the amino acid residue serine (S) from the third possible amino acid residue set 160c for inclusion at the corresponding position in the output protein sequence 150b. As described in more details below, in some cases, to identify the amino acid residues for inclusion in the output protein sequence 150b, the protein structure computation model 117 may select, from different possible permutations of amino acid residues formed from the amino acid residues included in the possible amino acid residue set 160 of each position in at least a portion of the output protein sequence 150b, a permutation of amino acid residues satisfying one or more criteria. In some cases, the one or more criteria may include an energy of the first protein structure 170 having the output protein sequence 150b, which may be determined by applying an energy function. That the possible permutations of amino acid residues are formed from the possible amino acid residue set 160 for each position in at least the portion of the output protein sequence 150b instead of, for example, every possible amino acid residue that can form an antibody, may drastically reduce the computation burden associated with identifying a permutation of amino acid residues for inclusion in the output protein sequence 150b such that the output protein sequence 150 and the corresponding first protein structure 170 exhibit certain desired properties.

At 304, the protein structure computation model 117 may determine a backbone conformation of a backbone of a protein structure having the protein sequence. In some example embodiments, the protein structure computation model 117 may further generate the first protein structure 170 by at least determining the conformation of the backbone of the first protein structure 170. In some cases, the backbone of the first protein structure 170 may include a continuous chain of atoms formed by linking a plurality of backbone atoms from each amino acid residue in the output protein sequence 150b. Moreover, in some cases, the plurality of backbone atoms in each amino acid residue in the output protein sequence may be a sequence of atoms containing a nitrogen (N) atom, an α-carbon (c_{α}) atom, and a carboxyl carbon (C) atom. It should be appreciated that the same plurality of backbone atoms may be present in different amino acid residues. In the example of the output protein sequence 150b shown in FIG. 6B, for instance, the backbone of the corresponding protein structure 170 may include the backbone atoms of each amino acid residue in the sequence containing arginine (R), alanine (A), serine(S), glutamine (Q), aspartic acid (D), valine (V), asparagine (N), threonine (T), alanine (A), valine (V), and alanine (A).

As noted, the protein structure computation model 117 may be applied to determine the backbone conformation of the first protein structure 170 with varying degrees of freedom. For instance, in some example embodiments, the protein structure computation model 117 may determine the first protein structure 170 based on the backbone of another protein structure, which may be associated with a protein sequence having one or more desired properties such expression, binding affinity towards a target molecule (e.g., an antigen such as a viral antigen or a tumor antigen), specificity towards the target molecule, lack of nonspecificity, stability, non-immunogenicity, human-ness, absence of self-association (or non-aggregation), and/or the like. In some cases, the other protein structure may have the input protein sequence 150a based on which the protein sequence computation model 113 generated the plurality of proposed sequences 155. Alternatively, the other protein structure may be associated with another protein sequence that is different from the input protein sequence 150a and the output protein sequence 150b. For instance, in some cases, the input protein sequence 150a, which is used to generate the plurality of proposed sequences 155 for determining the output protein sequence 150b, may be associated with a first desired property while the other protein sequence associated with the other protein structure providing the backbone conformation of the first protein structure 170 may be associated with the same first desired property and/or a second desired property. Accordingly, in some cases, the protein structure computation model 117 may determine a first backbone conformation of the backbone of the first protein structure 170 to have a same conformation as at least a portion of the backbone of the other protein structure such that the first protein structure 170 may exhibit the same (or similar) desired properties as the other protein structure. As described in more detail below, the protein design engine 110 may perform verification to determine if the output protein sequence 150b will fold into the first protein structure 170 having the backbone conformation of the other protein structure.

In some example embodiments, the protein design engine 110 may apply the protein structure computation model 117 to determine the backbone conformation of the first protein structure 170 independent of another protein structure. Instead, the protein structure computation model 117 may be applied to determine the geometric state of the backbone of the first protein structure 170 with varying degrees of freedom. For example, in some cases, the protein structure computation model 117 may determine the geometric state of the backbone of the first protein structure 170 by at least determining a translation and/or a rotation of at least a portion of the backbone atoms forming the backbone of the first protein structure 170. In some cases, in addition to or instead of the translation and/or the rotation of at least the portion of the backbone atoms in the backbone of the first protein structure 170, the protein structure computation model 117 may determine the geometric state of the backbone of the first protein structure 170 by at least determining a torsion angle of one or more rotatable bonds formed by at least a portion of the backbone atoms. For instance, in some cases, the geometric state of the backbone of the first protein structure 170 may be determined by determining one or more of the torsion angle ψ of the rotatable bond between the α-carbon (c_{α}) atom and the carbonyl group, the torsion angle ϕ of the rotatable bond between the α-carbon (c_{α}) atom and the nitrogen (N) atom, the torsion angle w of the rotatable bond between the carbon (C) atom and the nitrogen (N) atom, and/or the like.

At 306, the protein structure computation model 117 may generate the protein structure to include, at each position in at least a portion of the backbone, the amino acid residue selected for inclusion at a corresponding position in the protein sequence. In some example embodiments, the protein design engine 110 may apply the protein structure computation model 117 to further generate the first protein structure 170 by at least including, at each position in at least a portion of the backbone of the first protein structure 170, the sidechain atoms of the amino acid residue that is selected for inclusion in the corresponding position in the output protein sequence 150b. In the example shown in FIG. 6B, for instance, the protein structure computation model 117 may generate the first protein structure 170 by at least including, in the backbone of the first protein structure 170, the sidechain atoms of each of the amino acid residues arginine (R), alanine (A), serine(S), glutamine (Q), aspartic acid (D), valine (V), asparagine (N), threonine (T), alanine (A), valine (V), and alanine (A). As described in more detail below, the first protein structure 170 may be further determined by the protein design engine 110 applying the protein structure computation model 117 to determine the sidechain conformation of each amino acid residue selected for inclusion in the output protein sequence 150b.

At 308, the protein structure computation model 117 may determine a sidechain conformation of each amino acid residue selected for inclusion in the output protein sequence. In some example embodiments, the protein design engine 110 may apply the protein structure computation model 117 to further generate the first protein structure 170 having the output protein sequence 150b by at least determining the conformation of the sidechains of the first protein structure 170. In some cases, the sidechains of the first protein structure 170 may include, for each amino acid residue selected for inclusion in the output protein sequence 150b, the chemical group attached to the backbone of the first protein structure 170. In some cases, the sidechain of an amino acid residue, such as the amino acid residue arginine (R) selected to occupy the first position 600a of the output protein sequence 150b, may include one or more atoms (e.g., sidechain atoms) attached to the backbone atoms of the amino acid residue (e.g., the sequence of atoms containing a nitrogen (N) atom, an α-carbon (c_{α}) atom, and a carboxyl carbon (C) atom). In some cases, the sidechain of the amino acid residue may include one or more atoms (e.g., sidechain atoms) connected to the α-carbon (c_{α}) atom in the backbone of the amino acid residue.

In some cases, the protein structure computation model 117 may determine the sidechain conformations of the first protein structure 170 based at least on the backbone conformation of the first protein structure 170. Moreover, the protein structure computation model 117 may be applied to determine the sidechain conformations of the first protein structure 170 with varying degrees of freedom. For example, in some cases, the protein structure computation model 117 may determine the sidechain conformation of each amino acid residue in at least a portion of the output protein sequence 150b by at least selecting one of a plurality of possible rotamers associated with the amino acid residue, each of which corresponding to a single possible conformation of the constituent sidechain atoms. Determining the sidechain conformations of the first protein structure 170 in this manner limits each amino acid residue in the first protein structure 170 to one of several discrete structural variations, which may be more computationally efficient than determining the sidechain conformations across continuous structural variations. Alternatively, the protein structure computation model 117 may determine the sidechain conformation of each amino acid residue in at least a portion of the output protein sequence 150b by determining one or more of the translation of the sidechain, the rotation of the sidechain, and/or the torsion angle of one or more rotatable bonds formed by the sidechain atoms.

FIG. 3B depicts a flowchart illustrating an example of a process 350 for hybrid protein design, in accordance with some example embodiments. Referring to FIGS. 1-2 and 3A-3B, the process 350 may be performed by the protein design engine 110 to generate, based at least on the output protein sequence 150a, the output protein sequence 150b and the corresponding first protein structure 170. In some cases, the process 350 may implement operation 206 of the process 200 in which the protein structure computation model 117 selects the constituent amino acid residues of a protein sequence and determines the corresponding protein structure by at least determining the conformation of the backbone and sidechains of the protein structure. Moreover, in some cases, the process 350 may implement operation 302 of the process 300 in which the protein structure computation model 117 selects, for inclusion in the output protein sequence 150b, a plurality of amino acid residues satisfying one or more criteria such as the minimization of an energy function of the first protein structure 170 having the output protein sequence 150b.

At 352, the protein structure computation model 117 may determine a first energy of a first protein molecule having a first plurality of amino acid residues and a first conformation. In some example embodiments, the protein structure computation model 117 may generate the first protein structure 170 by at least determining a permutation of amino acid residues for inclusion in the output protein sequence 150b and a corresponding conformation (or spatial arrangement) of the constituent atoms having a minimal energy. In some cases, the protein structure computation model 117 may determine the permutation of amino acid residues for inclusion in the output protein sequence 150b by at least selecting, for each position in the output protein sequence 150b, an amino acid residue from the corresponding possible amino acid residue set 160 for inclusion in the output protein sequence 150b. Furthermore, in addition to determining the identities of the amino acid residues in each position of at least a portion of the output protein sequence 150b, the protein structure computation model 117 may determine the conformation of the corresponding first protein structure 170, for example, by determining the backbone and sidechain conformations of the first protein structure 170 having the output protein sequence 150b.

In some cases, the protein structure computation model 117 may determine the output protein sequence 150b and the first protein structure 170 formed by the output protein sequence 150b by at least generating multiple protein molecules with different sequences of amino acid residues and/or different conformations in order to identify a particular protein sequence and conformation satisfying one or more criteria. That is, the output protein sequence 150b and the corresponding protein structure 170 generated by the protein structure computation model 117 may satisfy one or more criteria such as the minimization of an energy of the first protein structure 170 having the output protein sequence 150b. Accordingly, in some cases, upon generating a first protein molecule having a first plurality of amino acid residues and a first conformation, the protein structure computation model 117 may determine the first energy of the first protein molecule, for example, by applying an energy function. In some cases, the protein structure computation model 117 may apply an energy function based on one or more of ab initio quantum mechanics, density functional theory (DFT), semiempirical methods, molecular mechanics force fields, statistical potentials, neural potentials, machine learning models (e.g., trained on structural data), and/or the like. For example, in some cases, the energy function for determining the first energy of the first protein molecule may be a physics-based energy function that determines, for the first protein molecule, a total energy including one or more of an electrostatic energy, covalent bonding energy, Van der Waals energy, and/or the like. It should be appreciated that different energy functions may be associated with different accuracy and computational complexity. For instance, a more accurate energy function, such as an ab initio quantum mechanics based energy function, may impose greater computational overhead than a less accurate energy function, such a molecular mechanics force fields based energy function. Accordingly, in some cases, the protein structure computation model 117 may apply a first energy function instead of a second energy function to determine the first energy of the first protein molecule based at least on a respective accuracy and/or computational complexity of the first energy function and the second energy function. As described in more details below, the protein structure computation model 117 may generate additional protein molecules with different sequences of amino acid residues and/or different conformations before identifying the one protein molecule having a lowest energy (e.g., total energy).

At 354, the protein structure computation model 117 may generate a second protein molecule having a second plurality of amino acid residues and a second conformation by modifying at least one of the first plurality of amino acid residues and the first conformation. In some example embodiments, the protein structure computation model 117 may generate one or mor additional protein molecules with different sequences of amino acid residues and/or different conformations than the first protein molecule generated in operation 352. For example, in some cases, the protein structure computation model 117 may generate a second protein molecule having at least one of a different sequence of amino acid residues and a different conformation than the first protein molecule generated in operation 352. In some cases, the second protein molecule may be generated by modifying the sequence of amino acid residues forming the first protein molecule, for example, by inserting, deleting, and/or modifying one or more of the amino acid residues in the first protein molecule. Alternatively and/or additionally, the second protein molecule may be generated by modifying the conformation of the first protein molecule, for example, by modifying one or more of a backbone conformation and a sidechain conformation of the first protein molecule.

At 356, the protein structure computation model 117 may determine a second energy of the second protein molecule. In some example embodiments, upon generating the second protein molecule having the second plurality of amino acid residues and the second conformation, the protein structure computation model 117 may determine the second energy of the second protein molecule, for example, by applying an energy function. In some cases, the energy function for determining the second energy of the second protein molecule may also be a physics-based energy function that determines, for the second protein molecule, a total energy including one or more of an electrostatic energy, covalent bonding energy, Van der Waals energy, and/or the like. For example, in some cases, the protein structure computation model 117 may determine the second energy of the second protein molecule by applying an energy function based on one or more of ab initio quantum mechanics, density functional theory (DFT), semiempirical methods, molecular mechanics force fields, statistical potentials, neural potentials, machine learning models (e.g., trained on structural data), and/or the like.

At 358, the protein structure computation model 117 may generate, based at least on the first energy being lower than the second energy, a protein structure having the first plurality of amino acid residues and the first conformation. In some example embodiments, the protein structure computation model 117 may generate the output protein sequence 150b of the first protein structure 170 to have the first plurality of amino acid residues forming the first protein molecule if the first energy of the first protein molecule is lower than the second energy of the second protein molecule. Furthermore, in instances where the first energy of the first protein molecule is lower than the second energy of the second protein molecule, the protein structure computation model 117 may generate the first protein structure 170 to have the first conformation of the first protein molecule. Moreover, in some cases, the protein structure computation model 117 may generate a third protein molecule having a third plurality of amino acid residues and a third conformation by modifying the first protein molecule (e.g., the first plurality of molecules and/or the first conformation) or the second protein molecule (e.g., the second plurality of molecules and/or the second conformation). In instances where the first energy of the first protein molecule is lower than the third energy of the third protein molecule, the protein structure computation model 117 may generate the first protein structure 170 to have the first conformation of the first protein molecule.

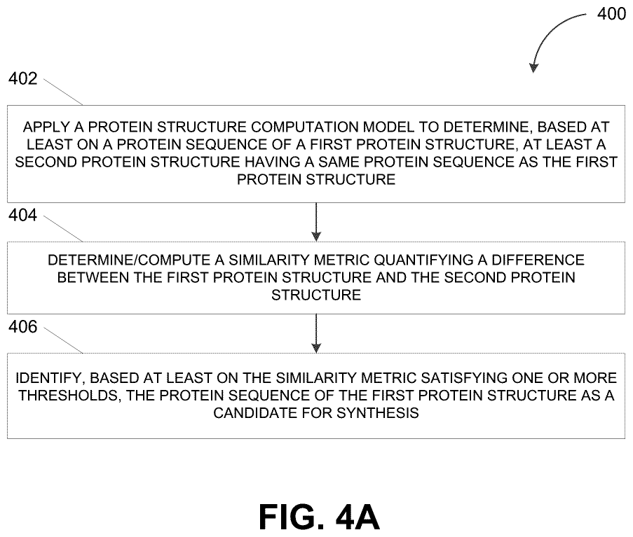

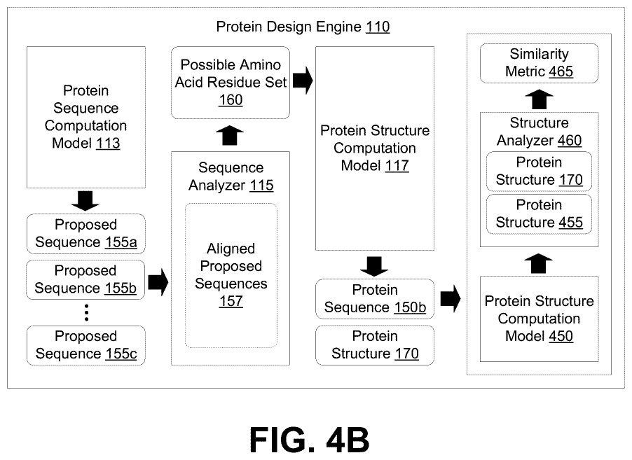

FIG. 4A depicts a flowchart illustrating another example of a process 400 for hybrid protein design, in accordance with some example embodiments. Referring to FIGS. 1-3A, 3B, and 4A, the process 400 may be performed by the protein design engine 110 to verify a protein structure generated by an in silico workflow. For example, in some cases, the protein design engine 110 may perform the process 400 to verify a protein structure generated by a protein structure computation model, such as the protein structure computation model 117 shown in FIG.

1. Alternatively and/or additionally, the protein design engine 110 may perform the process 500 to verify the protein structure generated by at least grafting a portion of one protein structure (e.g., a donor protein structure) onto a corresponding portion of another protein structure (e.g., a recipient or template protein structure).

At 402, the protein design engine 110 may apply a protein structure computation model to determine, based at least on a protein sequence of a first protein structure, at least a second protein structure having a same protein sequence as the first protein structure. In some example embodiments, where the protein design engine 110 applied the protein design computation model 117 to determine the first protein structure 170 having the output protein sequence 150b, the protein design engine 110 may apply a different protein structure computation model to verify that the output protein sequence 150b will fold into the first protein structure 170 (e.g., have the backbone and sidechain conformation of the first protein structure 170 determined by the protein design computation model 117). For instance, in the example shown in FIG. 4B, the protein design engine 110 may apply a protein structure computation model 450, which may be a different protein structure computation model than the protein structure computation model 117, to determine at least a second protein structure 455 formed by the output protein sequence 150b.

In some cases, the protein design engine 110 may apply the protein structure computation model 450 or multiple different protein structure computation models, to generate the second protein structure 455 or multiple protein structures formed by the output protein sequence 150b. Moreover, in some cases, the protein structure computation model 450 may differ from the protein structure computation model 117 at least because the protein structure computation model 450 includes a different machine learning model and/or implements a different structural design protocol than the protein structure computation model 117. For example, in some cases, where the protein structure computation model 117 determines the first protein structure 170 by at least determining the amino acid residues included in the output protein sequence 150b as well as the corresponding conformation (or spatial arrangement) of the amino acid residues (e.g., the constituent atoms in each amino acid residue), the protein structure computation model 450 may determine, based at least on the output protein sequence 150b, a second protein structure 465.

At 404, the protein design engine 110 may determine/compute a similarity metric quantifying a difference between the first protein structure and the second protein structure. In some example embodiments, the protein design engine 110 may include a structure analyzer 460 that determines a similarity metric 465 quantifying the difference between the first protein structure 170 generated by the protein structure computation model 117 and the second protein structure 455 generated by the protein structure computation model 450. Where the protein design engine 110 generates multiple protein structures associated with the output protein sequence 150b and not just the second protein structure 465, it should be appreciated that the structure analyzer 460 may determine the similarity metric 465 for each protein structure. In some cases, the similarity metric 465 may include a root mean square deviation (RMSD) computed based on the best-superimposed atomic coordinates of the first protein structure 170 and the second protein structure 455. However, it should be appreciated that the similarity metric 465 may include other values quantifying the structural differences between the first protein structure 170 and the second protein structure 455. For instance, in some cases, the structure analyzer 460 may perform principal component analysis (PCA), in which case the similarity metric 465 may include the correlation between the respective symmetric interaction matrix of the first protein structure 170 and the second protein structure 455. The symmetric interaction matrix of a protein structure may be constructed to include relationship parameters between secondary elements such as distance, orientation, and/or other relevant structural invariants.

At 406, the protein design engine 110 may identify, based at least on the similarity metric satisfying one more (preset) thresholds, the protein sequence of the first protein structure as a candidate for synthesis. In some example embodiments, where the similarity metric 465 quantifying the structural difference between the first protein structure 170 and the second protein structure 150b satisfies one or more thresholds, the protein design engine 110 may verify that the output protein sequence 150b will fold into the first protein structure 170 determined by the protein structure computation model 117. That is, the protein design engine 110 may verify that the first protein structure 170 has the actual conformation of the output protein sequence 150b if a different protein structure computation model, such as the protein structure computation model 450, determines a same (or sufficiently similar) protein structure for the output protein sequence 150b as the protein structure computation model 117. Where the protein design engine 110 generates multiple protein structures associated with the output protein sequence 150b and not just the output protein structure 465, the protein design engine 110 may verify that the first protein structure 170 has the actual conformation of the output protein sequence 150b if the similarity metric 465 of a threshold quantity of protein structures generated by a different protein structure computation model, such as the second protein structure 455 generated by the protein structure computation model 450, satisfies the one or more thresholds.

In instances where the first protein structure 170 (e.g., the specific conformation of the first protein structure 170) is associated with one or more desired properties, the output protein sequence 150b may be identified for further analysis including, for example, synthesis, in vitro measurements, in vivo characterizations, and/or the like, if the protein design engine 110 is able to verify that the output protein sequence 150b will fold into the conformation of the first protein structure 170. For example, the first protein structure 170 may exhibit one or more desired properties such as expression, binding affinity towards a target molecule (e.g., an antigen such as a viral antigen or a tumor antigen), specificity towards the target molecule, lack of nonspecificity, stability, non-immunogenicity, human-ness, absence of self-association (or non-aggregation), and/or the like. Accordingly, where the protein design engine 110 is able to verify that the output protein sequence 150b will fold into the first protein structure 170, the output protein sequence 150b is identified for further analysis at least because the protein design engine 110 may determine that the physical protein structures synthesized from the output protein sequence 150b are likely to exhibit the same desired properties as the first protein structure 170 generated in silico.

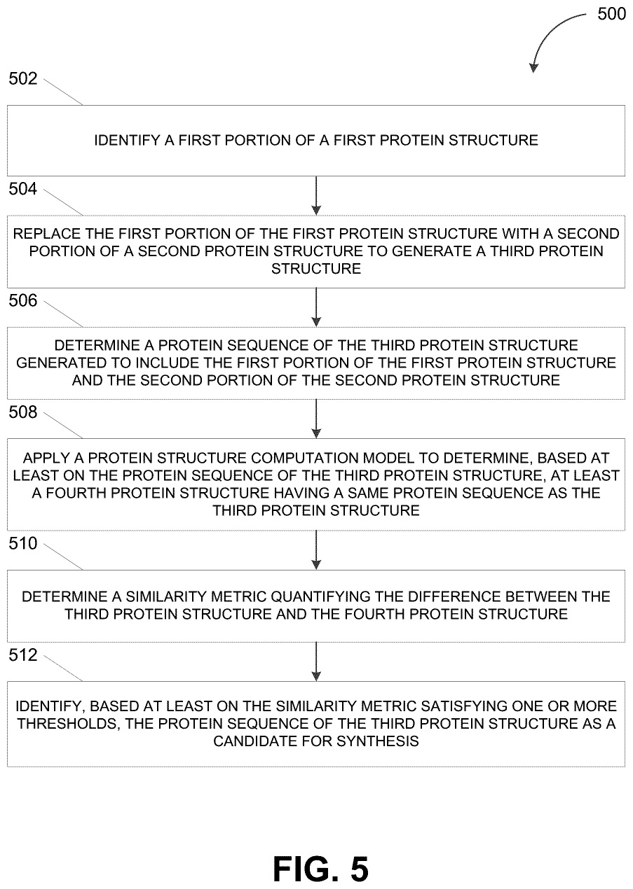

FIG. 5 depicts a flowchart illustrating another example of a process 500 for hybrid protein design, in accordance with some example embodiments. Referring to FIGS. 1-5, the process 500 may be performed by the protein design engine 110 to generate a protein structure by at least grafting a protein structure onto another protein structure. In some cases, the process 200 described in FIG. 2 and/or the process 300 described in FIG. 3A may be performed in order to generate the donor protein structure and/or the recipient (or template) protein structure used in the process 500. As described in more detail below, the donor protein structure may be a portion of a protein molecule (e.g., the antigen binding fragment (Fab), the paratope, the complementarity determining region (CDR), the variable region (Fv), and/or the like) that is grafted onto a corresponding portion of the recipient (or template) protein structure to form a complete protein molecule.

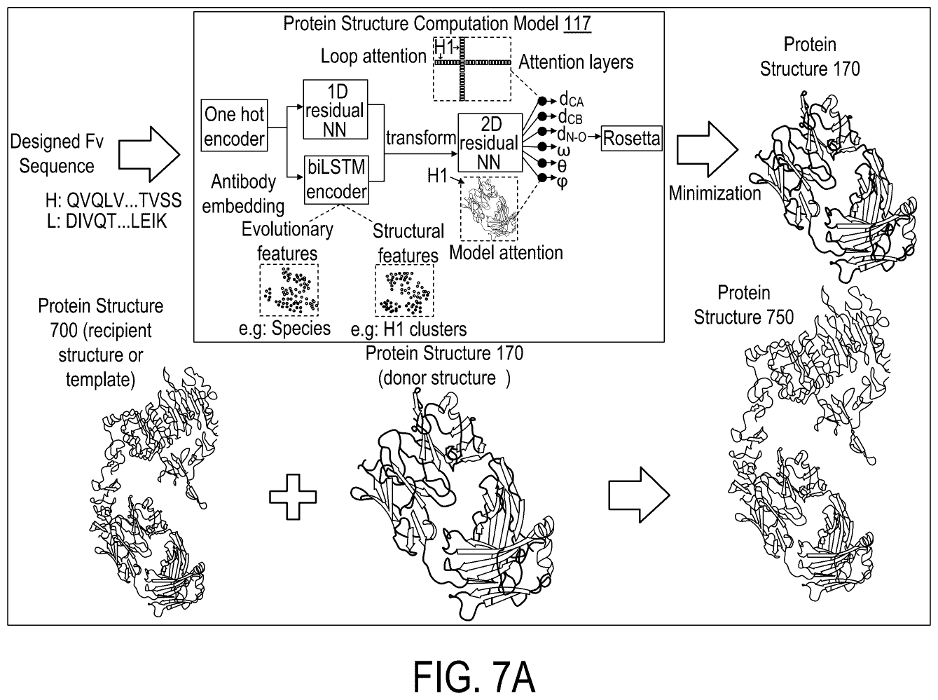

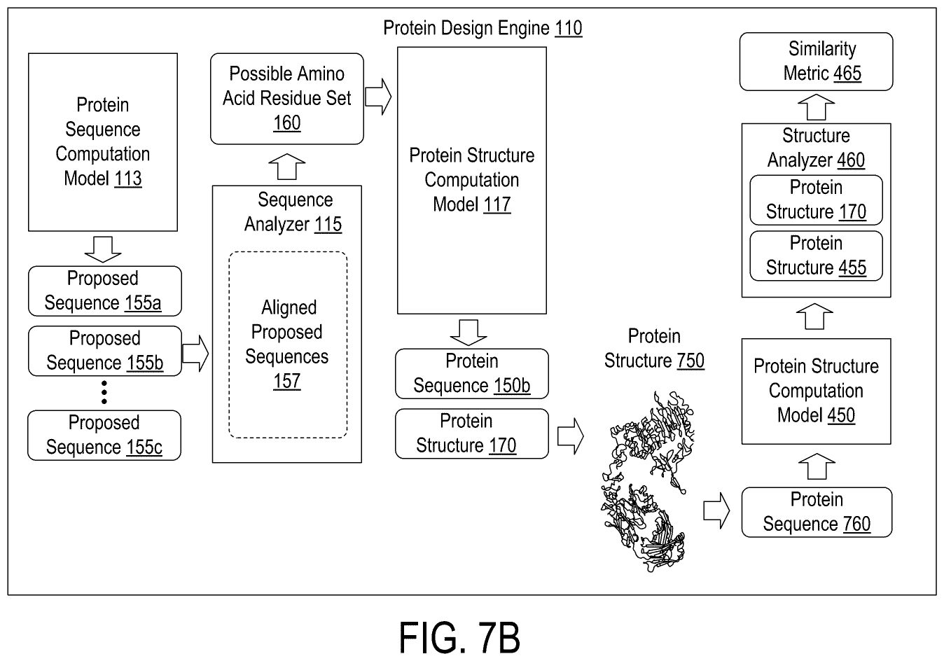

At 502, the protein design engine 110 may generate a first protein structure comprising at least a first portion of a protein molecule. As noted, in some example embodiments, the protein design engine 110 may apply the protein sequence computation model 113 and the protein structure computation model 117 to generate the first protein structure 170 having the output protein sequence 150b. In some instances, the first protein structure 170 generated by the protein sequence computation model 113 and the protein structure computation model 117 may be a donor structure including a portion of an entire protein molecule, such as the paratope, the variable region (Fv), the antigen binding fragment (Fab), or a complementarity determining region (CDR) of an antibody. For example, in some cases, the protein design engine 110 may apply the protein sequence computation model 113 and the protein structure computation model 117 to generate a particular portion of a protein molecule (and not the protein molecule in its entirety). In some cases, the aforementioned portion of the protein molecule may be one or more domains, each of which being an independently folding portion of the protein molecule. The first protein structure 170 serving as the donor structure may include the entire portion of the protein molecule generated by the protein sequence computation model 113 and the protein structure computation model 117 or, in some cases, a further subpart of that portion of the protein molecule. For instance, where the protein design engine 110 applies the protein sequence computation model 113 and the protein structure computation model 117 to generate the antigen binding fragment (Fab) of an antibody molecule, the first protein structure 170 may be the variable region (Fv) or one or more of the complementarity determining regions (CDRs) of the antigen binding fragment (Fab). Alternatively, the first protein structure 170 may be a portion of an entire protein molecule (e.g., an antibody and/or the like) generated by the protein sequence computation model 113 and the protein structure computation model 117. In instances where the protein sequence computation model 113 and the protein structure computation model 117 generates entire protein molecules, such as an entire antibody, the first protein structure 170 may be a portion that protein molecule, such as the paratope, the variable region (Fv), the antigen binding fragment (Fab), or a complementarity determining region (CDR) of an antibody, identified by the protein design engine 110 to serve as a donor structure. In the example shown in FIGS. 7A-B, the donor structure that includes at least a portion of the first protein structure 170 generated by the protein sequence computation model 113 and the protein structure computation model 117 may be grafted onto a second protein structure 700 (e.g., a recipient (or template) protein structure) to form a third protein structure 750, depicted in the embodiment shown at FIG. 7A.

At 504, the protein design engine 110 may replace a second portion of a second protein structure with the first protein structure to generate a third protein structure. In some example embodiments, the protein design engine 110 may replace a second portion the second protein structure 700 (e.g., the recipient (or template) protein structure) with the first protein structure 170 to generate the third protein structure 750. For example, in some cases, the first protein structure 170 may be a portion of a protein molecule, such as the paratope, the variable region (Fv), the antigen binding fragment (Fab), or a complementarity determining region (CDR) of an antibody. Accordingly, the protein design engine 110 may graft the first protein structure 170 onto the second protein structure 700 by at least replacing, with the first protein structure 170, a corresponding portion of the second protein structure 700. For instance, in instances where the first protein structure 170 is a first paratope, a first variable region (Fv), a first antigen binding fragment (Fab), or a first complementarity determining region (CDR) of a first antibody, the protein design engine 110 may replace, with the first protein structure 170, a second paratope, a second variable region (Fv), a second antigen binding fragment (Fab), or a second complementarity determining region (CDR) of a second antibody.

In some cases, the protein design engine 110 may graft the first protein structure 170 onto the second protein structure 700 to generate the third protein structure 750. In some cases, the third protein structure 750 may be a complete protein molecule (e.g., an antibody and/or the like) that combines portions of multiple protein structures including the first protein structure 170 and the second protein structure 700. For example in some cases, the third protein structure 750 may include the variable region (Fv) of the first protein structure 170 and the constant region (Fc) of the second protein structure 700. Alternatively and/or additionally, the third protein structure 750 may include the antigen binding fragment (Fab) of the first protein structure 170 and the crystallizable fragment (Fc) of the second protein structure 700. In some cases, the third protein structure 750 may include one or more complementarity determining regions (CDR) (or hypervariable regions) of the first protein structure 170 and one or more framework regions of the second protein structure 700.

At 506, the protein design engine 110 may determine a protein sequence of the third protein structure generated to include at least the first portion of the first protein structure and a third portion of the second protein structure. In some example embodiments, the protein design engine 110 may determine the protein sequence 760 of the third protein structure 750, which is a generated to include portions of the first protein structure 170 and the second protein structure 700, each being associated with a different underlying protein sequence. (Exemplary sequence 760 is depicted at FIG. 7B.) Accordingly, the protein sequence 760 of the third protein structure 750 may also include at least portions of the output protein sequence 150a of the first protein structure 170 and the protein sequence of the second protein structure 700. For example, in some cases, the protein sequence 760 the third protein structure 750 may include a first sequence of amino acid residues forming the variable region (Fv) of the first protein structure 170 and a second sequence of amino acid residues forming the constant region (Fc) of the second protein structure 700. Alternatively and/or additionally, the protein sequence 760 of the third protein structure 750 may include a first sequence of amino acid residues forming the antigen binding fragment (Fab) of the first protein structure 170 and a second sequence of amino acid residues forming the crystallizable fragment (Fc) of the second protein structure 700. In some cases, the protein sequence 760 of the third protein structure 750 may include a first sequence of amino acid residues forming one or more complementarity determining regions (CDR) (or hypervariable regions) of the first protein structure 170 and a second sequence of amino acid residues forming one or more framework regions of the second protein structure 700. As described in more detail below, in some cases, the protein design engine 110 may apply a protein structure computation model to verify that the protein sequence 760 of the third protein structure 750 will assume the conformation of the third protein structure 750 formed by grafting the first protein structure 170 (e.g., the donor structure) onto the second protein structure 700 (e.g., the recipient or template structure).

At 508, the protein design engine 110 may apply a protein structure computation model to determine, based at least on the protein sequence of the third protein structure, at least a fourth protein structure having a same protein sequence as the third protein structure. As noted, in some example embodiments, the protein design engine 110 may verify the third protein structure 750 formed by grafting the first protein structure 170 (e.g., the donor structure) onto the second protein structure 700 (e.g., the recipient or template structure) by at least verifying that the protein sequence 760 of the third protein structure 750, which includes sequences of amino acid residues from the first protein structure 170 and the second protein structure 700, will assume the conformation of the third protein structure 750. In some cases, the protein design engine 110 may verify the third protein structure 750 by applying one or more protein structure computation models 450 to determine, based at least on the protein sequence 760 of the third protein structure 750, one or more additional protein structures 455 for evaluation against the third protein structure 750. For example, in some cases, the protein design engine 110 may apply the protein structure computation model 117 used to generate the first protein structure 170 to generate the one or more additional protein structures 455 for verifying the third protein structure 750. Alternatively and/or additionally, the protein design engine 110 may apply the protein structure computation model used to generate the second protein structure 700 and/or another protein structure computation model to generate the one or more additional protein structures 455 for verifying the third protein structure 750. As described in more detail below, the verification of the third protein structure 750 may include evaluating the third protein structure 750 against the one or more additional protein structures 455 generated based on the protein sequence 760 of the third protein structure 750.

At 510, the protein design engine 110 may determine a similarity metric quantifying a difference between the third protein structure and the fourth protein structure. In some example embodiments, the structure analyzer 460 of the protein design engine 110 may determine the similarity metric 465 quantifying the difference between the third protein structure 750 and the one or more additional protein structures 455 generated based on the protein sequence 460 of the third protein structure 750. In some cases, the similarity metric 465 may include a root meat square deviation (RMSD), which quantifies the structural difference between the third protein structure 750 and each of the additional protein structures 455 generated based on the protein sequence 760 of the third protein structure 750. Moreover, in some cases, the similarity metric 465 may indicate the likelihood that the protein sequence 760 of the third protein structure 750 will assume the conformation of the third protein structure 750, which is generated by grafting the first protein structure 170 onto the second protein structure 700.

At 512, the protein design engine 110 may identify, based at least on the similarity metric satisfying one or more thresholds, the protein sequence of the third protein structure as a candidate for synthesis. In some example embodiments, where the similarity metric 465 quantifying the structural difference between the third protein structure 750 and the one or more additional protein structures 455 satisfies one or more thresholds, the protein design engine 110 may verify that the protein sequence 760 will fold into the third protein structure 750 generated by grafting the first protein structure 170 onto the second protein structure 700. In some cases, the protein design engine 110 may verify that the third protein structure 750 exhibits the actual conformation of the protein sequence 760 if the one or more protein structure computation models 450 determines a same (or sufficiently similar) protein structure for the protein sequence 760 as the first proteins structure 170 grafted onto the second protein structure 700. Where the protein design engine 110 generates multiple protein structures 455 having the protein sequence 760 of the third protein structure 750, the protein design engine 110 may verify that the third protein structure 750 has the actual conformation of the protein sequence 760 if the similarity metric 465 of a threshold quantity of the additional protein structures 455 satisfies the one or more thresholds.

In some example embodiments, upon verifying the third protein structure 750, the protein design engine 110 may select the corresponding protein sequence 760 for further analysis including, for example, synthesis, in vitro measurements, in vivo characterizations, and/or the like. For example, in some cases, the third protein structure 760 or one or more of its constituent components, such as the first protein structure 170 and/or the second protein structure 700, may exhibit one or more desired properties such as expression, binding affinity towards a target molecule (e.g., an antigen such as a viral antigen or a tumor antigen), specificity towards the target molecule, lack of nonspecificity, stability, non-immunogenicity, human-ness, absence of self-association (or non-aggregation), and/or the like. Accordingly, where the protein design engine 110 is able to verify that the protein sequence 760 will fold into the third protein structure 750, the protein design engine 110 may identify the protein sequence 760 of the third protein structure 750 for further analysis at least because the protein design engine 110 may determine that the physical protein structures synthesized from the protein sequence 760 are likely to exhibit the same desired properties as the third protein structure 760. For instance, where the first protein structure 170 is a paratope, an antigen binding fragment (Fab), a variable region (Fv), or a complementarity determining region (CDR) that exhibits binding affinity towards a target molecule and the third protein structure 750 is generated to include the first protein structure 170 grafted onto the second protein structure 700 as the corresponding constant region, crystallizable fragment (Fc), or framework regions of the third protein structure 750, the protein design engine 110 may determine that the protein structures synthesized from the protein sequence 760 of the third protein structure 750 are likely to exhibit the same binding affinity towards the target molecule.

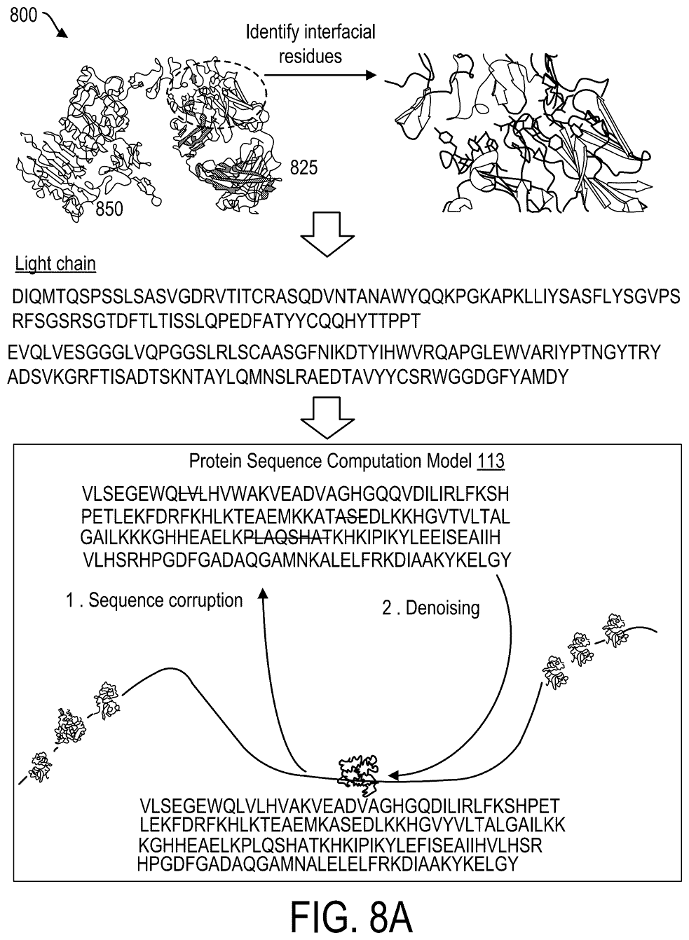

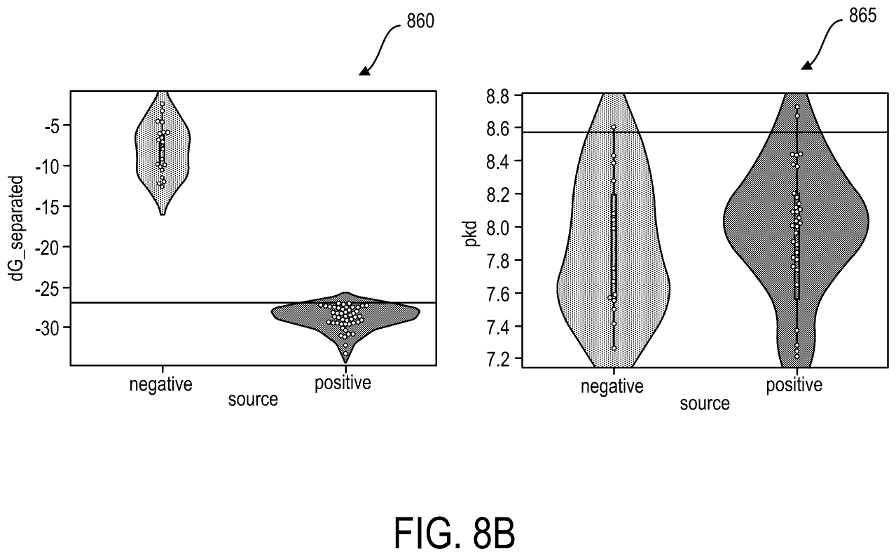

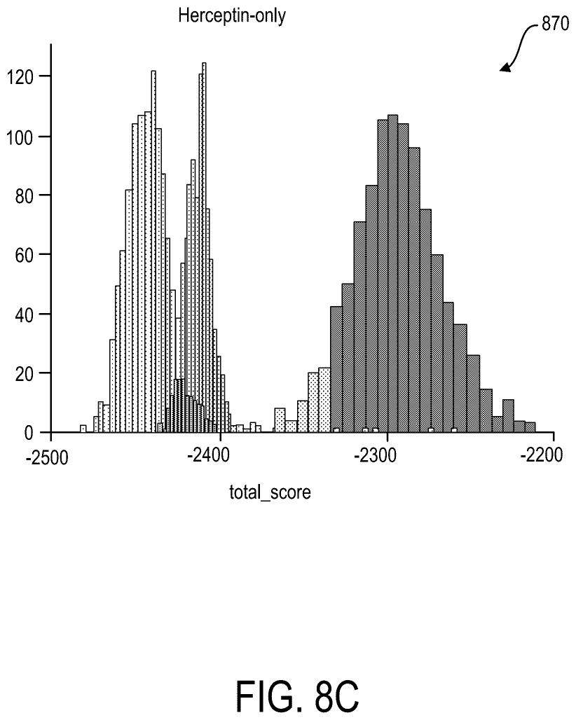

FIG. 8A depicts a schematic diagram illustrating an example of a process for hybrid protein design in which an alternate protein sequence is generated for the antigen binding fragment of the antibody trastuzumab, in accordance with some example embodiments. Referring to FIG. 8A, the protein design engine 110 may determine, based at least on a co-crystal structure 800 of a first protein structure 825 corresponding to the antibody trastuzumab bound to a second protein structure 850 corresponding to the protein human epidermal growth factor receptor 2 (HER2), one or more interfacial residues. In this context, an interfacial residue may refer to an amino acid residue in one protein molecule, such as the first protein structure 825 of the antibody molecule trastuzumab, that interacts and contacts with another protein molecule, such as the second protein structure 850 of the human epidermal growth factor receptor 2 (HER2) protein molecule. As shown in FIG. 8A, the interfacial residues of the antibody trastuzumab may include the constituent amino acid residues of the antigen binding fragment (Fab) of the antibody trastuzumab. In some cases, the interfacial residues of the antibody trastuzumab may include the amino acid residues forming variable domains of the light chain (V_{L}) and the heavy chain (V_{H}) of the antibody trastuzumab.

Referring again to FIG. 8A, in some example embodiments, one or more alternate protein sequences for the antibody trastuzumab (or certain portions thereof) may be generated by at least applying the protein sequence computation model 113. In the example shown in FIG. 8A, the protein sequence computation model 113 may be an autoencoder (e.g., a variational autoencoder and/or the like) that generates each alternate protein sequence for the antibody trastuzumab by sampling a data distribution occupied by encodings of known protein sequences. Accordingly, for each sampling iteration of the data distribution, the protein sequence computation model 113 may encode at least a portion of the original protein sequence of the antibody trastuzumab before decoding an intermediate sequence having at least one of a corruption (e.g., an insertion, a deletion, and/or a modification of an amino acid residue) and a length change relative to the protein sequence of trastuzumab.

In some cases, the protein design engine 110 may apply the protein sequence computation model 113 to generate alternate sequences for a portion of the antibody trastuzumab (e.g., the paratope, the antigen binding fragment (Fab), the variable region (Fv), the complementarity determining region (CDR), and/or the like). Alternatively, the protein design engine 110 may apply the protein sequence computation model 113 to generate alternate sequences for the entire trastuzumab molecule. For example, in some cases, the protein design engine 110 may apply the protein sequence computation model 113 to generate multiple proposed protein sequences based on the original protein sequence of the antibody trastuzumab (or a portion thereof) before determining, based on the proposed protein sequences, a possible amino acid residue set for each position in the alternate trastuzumab protein sequence. In some cases, the possible amino acid residue set for a position in the alternate trastuzumab protein sequence may include one or more amino acid residues that appear with sufficient frequency in that position across the proposed protein sequences generated by the protein sequence computation model 113.

In some example embodiments, the alternate protein sequence for the antibody trastuzumab (or a portion thereof) may be generated by applying the protein structure computation model 117, which determines the alternate trastuzumab sequence based on the possible amino acid residue set for each position as well as the corresponding conformation of the protein molecule having the alternate protein sequence. In cases where the conformation assumed by the alternate protein sequence is sufficiently similar to that of the original trastuzumab molecule (e.g., the first protein structure 825 in the co-crystal structure 800), the protein design engine 110 may identify the alternate protein sequence for further analysis including, for example, synthesis, in vitro measurements, in vivo characterizations, and/or the like. For example, the protein design engine 110 may apply a protein structure computation model, which may or may not be the protein structure computation model 117 applied to generate the alternate sequence and structure of trastuzumab, to determine the conformation of the protein molecule having the alternate protein sequence. Where the conformation of the protein molecule having the alternate protein sequence exhibits sufficient similarity to the original structure of trastuzumab, such as when a similarity metric (e.g., a root mean square deviation (RMSD) and/or the like) quantifying the difference between the two molecules satisfies one or more thresholds, the protein design engine 110 may identify the alternate sequence for trastuzumab for further analysis (e.g., synthesis, in vitro measurements, in vivo characterizations, and/or the like).

FIG. 8B depicts a violin graph 860 illustrating the predicted binding affinity of the alternate trastuzumab sequence generated by different hybrid protein design workflows and a violin graph 865 illustrating the measured affinity, in accordance with some example embodiments. Referring to FIG. 8B, the protein structure generated by each hybrid protein design workflow may be evaluated by computing the interface energy (dG separated), which indicates the predicted binding affinity of each alternate trastuzumab protein sequence (to the human epidermal growth factor receptor 2 (HER2)) by at least quantifying the difference between a first energy of the bound trastuzumab human epidermal growth factor receptor 2 (HER2) protein complex and a second energy of the separated protein molecules. The binding affinity of each alternate trastuzumab protein sequence may also be determined empirically by measuring, for example, the dissociation constant (K_{D}) of the bound trastuzumab human epidermal growth factor receptor 2 (HER2) protein complex. The violin graphs 860 and 865 show, respectively, the distribution of the interface energy (dG separated) and the measured binding affinity (pkd) across a first population of nonbinding alternate trastuzumab protein sequences (labeled negative) and a second population of binding alternate trastuzumab protein sequences. These two populations of alternate trastuzumab protein sequences were generated by a hybrid protein design workflow that includes a protein sequence computation model trained based on a training dataset containing a first plurality of labeled nonbinding protein sequences and a second plurality of labeled binding protein sequences. As the graphs 850 and 860 show, a large proportion of the binding alternate trastuzumab protein sequences generated by the hybrid protein design workflow exhibit a high binding affinity, as indicated by the higher dissociation constant and lower interface energy.

FIG. 8C depicts a graph 870 illustrating the stability of each alternate trastuzumab protein structure, as indicated by the total energy of the molecule. The graph 870 in FIG. 8C shows that the alternate trastuzumab protein sequences generated by various hybrid protein design workflows described herein fold into more stable three-dimensional structure (e.g., with lower total energy) than conventional structurally agnostic protein sequence design techniques.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A computer-implemented method, comprising: identifying a protein sequence computation model and a protein structure computation model; applying the protein sequence computation model to generate, based at least on an input protein sequence, a plurality of proposed protein sequences; identifying, based at least on the plurality of proposed protein sequences, a set of possible amino acid residues for each position in at least a portion of an output protein sequence; and generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.

Item 2: The method of Item 1, further comprising: aligning the plurality of proposed protein sequences to generate an aligned plurality of protein sequences; and identifying, based at least on the aligned plurality of protein sequences, the set of possible amino acid residues for each position in at least the portion of the output protein sequence.

Item 3: The method of any of Items 1 to 2, wherein the plurality of proposed protein sequences are aligned by applying one or more of dynamic programming, progressive alignment, hierarchical alignment, iterative alignment, motif finding, a deep learning model, and a Hidden Markov model.

Item 4: The method of any of Items 1 to 4, wherein the identifying of the set of possible amino acid residues for each position in at least the portion of the output protein sequence includes identifying, for inclusion in the set of possible amino acid residues, a first amino acid residue but not a second amino acid residue.

Item 5: The method of Item 4, wherein the identifying of the set of possible amino acid residues for each position in at least the portion of the output protein sequence further includes determining a first frequency at which a first amino acid residue appears at the position across the plurality of proposed protein sequences generated by the protein sequence computation model, determining a second frequency at which a second amino acid residue appears at the position across the plurality of proposed protein sequences generated by the protein sequence computation model, and identifying, based at least on the first frequency and the second frequency, the first amino acid residue but not the second amino acid residue for inclusion in the set of possible amino acid residues for the position.

Item 6: The method of Item 5, wherein the first amino acid residue identified for inclusion in the set of amino acid residues based at least on the first frequency satisfying one or more thresholds, and wherein the second amino acid residue identified for exclusion from the set of possible amino acid residues based at least on the second frequency failing to satisfy the one or more thresholds.

Item 7: The method of Item 6, wherein the identifying of the set of possible amino acid residues for the position in the output protein sequence further includes determining the one or more thresholds based on at least one of a maximum, a minimum, a median, a mean, and a mode of a frequency at which each of a plurality of amino acid residues appear at the position across the plurality of proposed protein sequences generated by the protein sequence computation model.

Item 8: The method of any of Items 1 to 7, wherein the set of possible amino acid residues includes some but not all of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, and pyrrolysine.

Item 9: The method of any of Items 1 to 8, wherein the protein structure computation model generates the first protein structure by at least determining, based at least on an energy of the first protein structure having the output protein sequence, an identity and a conformation of an amino acid residue occupying each position in at least the portion of the output protein sequence.

Item 10: The method of Item 9, wherein the protein structure computation model determines the identity and the conformation of the amino acid residue occupying each position in at least the portion of the output protein sequence by at least modifying at least one of the identity and the conformation of the amino acid residue to minimize an energy of the first protein structure.

Item 11: The method of Item 10, wherein the protein structure computation model modifies at least one of the identity and the conformation of the amino acid residue occupying a position by at least (i) changing a conformation of an amino acid residue occupying the position or (ii) selecting, from the set of amino acid residue associated with the position, a different possible amino acid residue for the position.

Item 12: The method of any of Items 9 to 11, wherein the protein structure computation model determines the identity and the conformation of the amino acid residue occupying each position in at least the portion of the output protein sequence by at least determining a first energy of the first protein structure having a first possible amino acid residue from the set of possible amino acid residues, determining a second energy of the first protein structure having a second possible amino acid residue from the set of possible amino acid residues, and generating the first protein structure to include, based at least on the first energy being lower than the second energy, the first possible amino acid residue instead of the second possible amino acid residue.

Item 13: The method of Item 12, wherein the protein structure computation model further determines the identity and the conformation of the amino acid residue occupying each position in at least the portion of the output protein sequence by at least determining a third energy of the first protein structure having a first conformation of the first possible amino acid residue, determining a fourth energy of the first protein structure having a second conformation of the first possible amino acid residue, and generating the first protein structure to include, based at least on the third energy being lower than the fourth energy, the first conformation of the first possible amino acid residue instead of the second conformation of the first possible amino acid residue.

Item 14: The method of any of Items 1 to 14, wherein the protein structure computation model generates the first protein structure by at least determining a first backbone conformation of a first backbone of the first protein structure having the output protein sequence.

Item 15: The method of Item 14, wherein the first backbone of the first protein structure is a continuous chain of atoms formed by linking a plurality of backbone atoms from each amino acid residue in the output protein sequence.

Item 16: The method of Item 15, wherein the plurality of backbone atoms is a sequence of atoms containing a nitrogen (N) atom, an a-carbon (ca) atom, and a carboxyl carbon (C) atom.

Item 17: The method of any of Items 14 to 16, wherein the determining the first backbone conformation of the first backbone of the first protein structure includes determining the first backbone of the first protein structure to have a same conformation as at least a portion of a second backbone of a second protein structure.

Item 18: The method of Item 17, wherein the second protein structure is associated with a protein sequence determined to have one or more desired properties.

Item 19: The method of Item 18, wherein the protein sequence having the one or more desired properties is the input protein sequence or another protein sequence.

Item 20: The method of any of Item 17 to 19, wherein the second backbone of the second protein structure exhibits a second backbone conformation of the second protein structure in an unbound state or a third backbone conformation of the second protein structure bound to a target molecule.

Item 21: The method of any of Items 14 to 20, wherein the determining the first backbone conformation of the first backbone of the first protein structure includes determining a translation of the plurality of backbone atoms included in each amino acid residue in at least a portion of the output protein sequence.

Item 22: The method of any of Items 14 to 21, wherein the determining of the first backbone conformation of the first protein structure includes determining a rotation of the plurality of backbone atoms included in each amino acid residue in at least a portion of the output protein sequence.

Item 23: The method of any of Items 14 to 22, wherein the determining of the first backbone conformation of the first protein structure includes determining a torsion angle of one or more rotatable bonds formed by the plurality of backbone atoms included in each amino acid residue in the output protein sequence.

Item 24: The method of any of Items 14 to 23, wherein the protein structure computation model further generates the first protein structure to include, at each position in at least a portion of the first backbone having the first backbone conformation, a plurality of sidechain atoms of the amino acid residue selected for inclusion at a corresponding position in the output protein sequence.

Item 25: The method of Item 24, further comprising: applying a different protein structure computation model to determine, based at least on the output protein sequence, at least a second protein structure having the output protein sequence; and determining a similarity metric quantifying a difference between the second protein structure and the first protein structure generated to have the first backbone conformation.

Item 26: The method of Item 25, further comprising: identifying, based at least on the similarity metric satisfying one or more thresholds, the output protein sequence as a candidate for synthesis.

Item 27: The method of any of Items 14 to 26, wherein the protein structure computation model further generates the first protein structure by at least determining, for each position in at least a portion of the first backbone having the first backbone conformation, a sidechain conformation of the amino acid residue selected for inclusion at a corresponding position in the output protein sequence.

Item 28: The method of Item 27, wherein the sidechain conformation of the amino acid residue is determined based at least on the first backbone conformation of the first backbone of the first protein structure.

Item 29: The method of any of Items 27 to 28, wherein the determining of the sidechain conformation of the amino acid residue selected for inclusion in the output protein sequence includes selecting a rotamer from a plurality of possible rotamers.

Item 30: The method of Item 29, wherein each rotamer in the plurality of possible rotamers includes a different combination of torsion angles of one or more rotatable bonds formed by a plurality of sidechain atoms in the amino acid residue.

Item 31: The method of any of Items 27 to 30, wherein the determining of the sidechain conformation of the amino acid residue selected for inclusion in the output protein sequence includes determining a torsion angle of one or more rotatable bonds formed by a plurality of sidechain atoms in the amino acid residue.

Item 32: The method of any of Items 27 to 31, wherein the determining of the sidechain conformation of the amino acid residue selected for inclusion in the output protein sequence includes determining a translation of a plurality of sidechain atoms in the amino acid residue.

Item 33: The method of any of Items 27 to 32, wherein the determining of the sidechain conformation of the amino acid residue selected for inclusion in the output protein sequence includes determining a rotation of a plurality of sidechain atoms in the amino acid residue.

Item 34: The method of any of Items 1 to 33, further comprising: applying a property analysis model to determine a property of each protein sequence included in the plurality of proposed protein sequences; identifying, based at least on the property of each protein sequence, at least one protein sequence in the plurality of proposed protein sequences for exclusion; and excluding, from the plurality of proposed protein sequences, the at least one protein sequence prior to identifying, based at least on a remaining plurality of proposed protein sequences, the set of possible amino acid residues for each position in the output protein sequence.

Item 35: The method of any of Items 1 to 34, further comprising: identifying a first portion of a second protein structure; and generating a third protein structure by at least replacing the first portion of the second protein structure with at least a second portion of the first protein structure.

Item 36: The method of Item 35, further comprising: determining a third protein sequence of the third protein structure generated to include the second portion of the first protein structure and a third portion of the second protein structure; applying the protein structure computation model and/or a different protein structure computation model to determine, based at least on the third protein sequence, at least a fourth protein structure having the third protein sequence; determining a similarity metric quantifying a difference between the third protein structure and the fourth protein structure; and identifying, based at least on the similarity metric satisfying one or more thresholds, the third protein sequence as a candidate for synthesis.

Item 37: The method of any of Items 35 to 36, wherein the second protein structure is selected based at least on the second protein structure exhibiting one or more desired properties.

Item 38: The method of any of Items 35 to 37, wherein the first portion of the second protein structure includes a first antigen binding site of a first antibody having the second protein structure, and wherein the second portion of the first protein structure includes a second antigen binding site of a second antibody having the first protein structure.

Item 39: The method of any of Items 35 to 38, wherein the first portion of the second protein structure includes a first paratope of a first antibody having the second protein structure, and wherein the second portion of the first protein structure includes a second paratope of a second antibody having the first protein structure.

Item 40: The method of any of Items 35 to 39, wherein the first portion of the second protein structure includes a first complementarity determining region (CDR) of a first antibody having the second protein structure, and wherein the second portion of the first protein structure includes a second complementarity determining region (CDR) of a second antibody having the first protein structure.

Item 41: The method of any of Items 1 to 40, wherein the protein structure computation model generates the first protein structure by at least determining, based at least on one or more criteria, a plurality of amino acid residues for inclusion in the output protein sequence and a corresponding conformation of the plurality of amino acid residues.

Item 42: The method of Item 41, wherein the one or more criteria include minimizing an energy function of the first protein structure having the plurality of amino acid residues selected for inclusion in the output protein sequence.

Item 43: The method of any of Items 41 to 42, wherein protein structure computation model generates the first protein structure by at least determining a first energy of a first protein molecule having a first plurality of amino acid residues and a first conformation; generating a second protein molecule having a second plurality of amino acid residues and a second conformation by modifying at least one of the first plurality of amino acid residues and the first conformation; determining a second energy of the second protein molecule; and generating, based at least on the first energy being lower than the second energy, the first protein structure to have the first plurality of amino acid residues and the first conformation.

Item 44: The method of any of Items 1 to 43, wherein the protein sequence computation model includes one or more machine learning models trained to generate the plurality of proposed protein sequences based on the input protein sequence.

Item 45: The method of any of Items 1 to 44, wherein the protein sequence computation model generates the plurality of proposed protein sequences by at least sampling a data distribution populated by a plurality of encoded protein sequences, and wherein each sampling of the data distribution generates an encoding of a protein sequence having at least one of a corruption and a length change relative to the input protein sequence.

Item 46: A system, comprising: at least one data processor; and at least one memory storing instructions, which when executed by the at least one data processor, result in operations comprising the method of any of Items 1 to 45.

Item 47: A non-transitory computer readable medium storing instructions, which when executed by at least one data processor, result in operations comprising the method of any of Items 1 to 45.

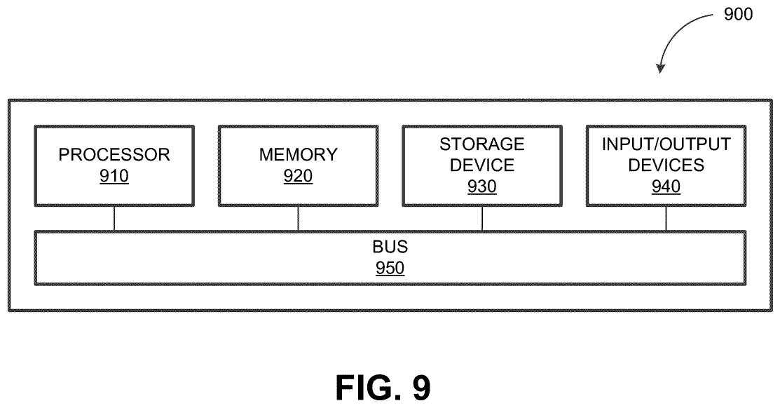

FIG. 9 depicts a block diagram illustrating an example of computing system 900, in accordance with some example embodiments. Referring to FIGS. 1-9, the computing system 900 may be used to implement the protein design engine 110, the molecular analysis engine 120, the client device 130, and/or any components therein.

As shown in FIG. 9, the computing system 900 can include a processor 910, a memory 920, a storage device 930, and input/output devices 940. The processor 910, the memory 920, the storage device 930, and the input/output devices 940 can be interconnected via a system bus 950. The processor 910 is capable of processing instructions for execution within the computing system 900. Such executed instructions can implement one or more components of, for example, the protein design engine 110, the analysis engine 120, the client device 130, and/or the like. In some example embodiments, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a hard disk device, an optical disk device, or a tape device, or other suitable persistent storage means. The input/output device 940 provides input/output operations for the computing system 900. In some example embodiments, the input/output device 940 includes a keyboard and/or pointing device. In various implementations, the input/output device 940 includes a display unit for displaying graphical user interfaces.

According to some example embodiments, the input/output device 940 can provide input/output operations for a network device. For example, the input/output device 940 can include Ethernet ports or other networking ports to communicate with one or more wired and/or wireless networks (e.g., a local area network (LAN), a wide area network (WAN), the Internet).

In some example embodiments, the computing system 900 can be used to execute various interactive computer software applications that can be used for organization, analysis and/or storage of data in various formats. Alternatively, the computing system 900 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desired results. Other implementations may be within the scope of the following claims.

1. A system, comprising:
at least one data processor; and
at least one memory storing instructions, which when executed by the at least one data processor, result in operations comprising:
identifying a protein sequence computation model and a protein structure computation model;
applying the protein sequence computation model to generate, based at least on an input protein sequence, a plurality of proposed protein sequences;
identifying, based at least on the plurality of proposed protein sequences, a set of possible amino acid residues for each position in at least a portion of an output protein sequence; and
generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.

2. The system of claim 1, wherein the operations further comprise:
aligning the plurality of proposed protein sequences to generate an aligned plurality of protein sequences; and
identifying, based at least on the aligned plurality of protein sequences, the set of possible amino acid residues for each position in at least the portion of the output protein sequence.

3. The system of claim 1, wherein the plurality of proposed protein sequences are aligned by applying one or more of dynamic programming, progressive alignment, hierarchical alignment, iterative alignment, motif finding, a deep learning model, and a Hidden Markov model.

4. The system of claim 1, wherein the identifying of the set of possible amino acid residues for each position in at least the portion of the output protein sequence includes identifying, for inclusion in the set of possible amino acid residues, a first amino acid residue but not a second amino acid residue.

5. The system of claim 4, wherein the identifying of the set of possible amino acid residues for each position in at least the portion of the output protein sequence further includes
determining a first frequency at which a first amino acid residue appears at the position across the plurality of proposed protein sequences generated by the protein sequence computation model,
determining a second frequency at which a second amino acid residue appears at the position across the plurality of proposed protein sequences generated by the protein sequence computation model, and
identifying, based at least on the first frequency and the second frequency, the first amino acid residue but not the second amino acid residue for inclusion in the set of possible amino acid residues for the position.

6. The system of claim 5, wherein the first amino acid residue is identified for inclusion in the set of amino acid residues based at least on the first frequency satisfying one or more thresholds, and wherein the second amino acid residue is identified for exclusion from the set of possible amino acid residues based at least on the second frequency failing to satisfy the one or more thresholds.

7. The system of claim 6, wherein the identifying of the set of possible amino acid residues for the position in the output protein sequence further includes
determining the one or more thresholds based on at least one of a maximum, a minimum, a median, a mean, and a mode of a frequency at which each of a plurality of amino acid residues appear at the position across the plurality of proposed protein sequences generated by the protein sequence computation model.

8. The system of claim 1, wherein the set of possible amino acid residues includes some but not all of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, and pyrrolysine.

9. The system of claim 1, wherein the protein structure computation model generates the first protein structure by at least determining, based at least on an energy of the first protein structure having the output protein sequence, an identity and a conformation of an amino acid residue occupying each position in at least the portion of the output protein sequence.

10. The system of claim 9, wherein the protein structure computation model determines the identity and the conformation of the amino acid residue occupying each position in at least the portion of the output protein sequence by at least modifying at least one of the identity and the conformation of the amino acid residue to minimize an energy of the first protein structure.

11. The system of claim 10, wherein the protein structure computation model modifies at least one of the identity and the conformation of the amino acid residue occupying a position by at least (i) changing a conformation of an amino acid residue occupying the position or (ii) selecting, from the set of amino acid residue associated with the position, a different possible amino acid residue for the position.

12. The system of claim 9, wherein the protein structure computation model determines the identity and the conformation of the amino acid residue occupying each position in at least the portion of the output protein sequence by at least
determining a first energy of the first protein structure having a first possible amino acid residue from the set of possible amino acid residues,
determining a second energy of the first protein structure having a second possible amino acid residue from the set of possible amino acid residues, and
generating the first protein structure to include, based at least on the first energy being lower than the second energy, the first possible amino acid residue instead of the second possible amino acid residue.

13. The system of claim 12, wherein the protein structure computation model further determines the identity and the conformation of the amino acid residue occupying each position in at least the portion of the output protein sequence by at least
determining a third energy of the first protein structure having a first conformation of the first possible amino acid residue,
determining a fourth energy of the first protein structure having a second conformation of the first possible amino acid residue, and
generating the first protein structure to include, based at least on the third energy being lower than the fourth energy, the first conformation of the first possible amino acid residue instead of the second conformation of the first possible amino acid residue.

14. The system of claim 1, further comprising:
applying a property analysis model to determine a property of each protein sequence included in the plurality of proposed protein sequences;
identifying, based at least on the property of each protein sequence, at least one protein sequence in the plurality of proposed protein sequences for exclusion; and
excluding, from the plurality of proposed protein sequences, the at least one protein sequence prior to identifying, based at least on a remaining plurality of proposed protein sequences, the set of possible amino acid residues for each position in the output protein sequence.

15. The system of claim 1, further comprising:
identifying a first portion of a second protein structure; and
generating a third protein structure by at least replacing the first portion of the second protein structure with at least a second portion of the first protein structure.

16. The system of claim 15, further comprising:
determining a third protein sequence of the third protein structure generated to include the second portion of the first protein structure and a third portion of the second protein structure;
applying the protein structure computation model and/or a different protein structure computation model to determine, based at least on the third protein sequence, at least a fourth protein structure having the third protein sequence;
determining a similarity metric quantifying a difference between the third protein structure and the fourth protein structure; and
identifying, based at least on the similarity metric satisfying one or more thresholds, the third protein sequence as a candidate for synthesis.

17. The system of claim 15, wherein the second protein structure is selected based at least on the second protein structure exhibiting one or more desired properties.

18. The method system of claim 15, wherein the first portion of the second protein structure includes a first antigen binding site of a first antibody having the second protein structure, and wherein the second portion of the first protein structure includes a second antigen binding site of a second antibody having the first protein structure.

19. The system of claim 15, wherein the first portion of the second protein structure includes a first paratope of a first antibody having the second protein structure, and wherein the second portion of the first protein structure includes a second paratope of a second antibody having the first protein structure.

20. The system of claim 15, wherein the first portion of the second protein structure includes a first complementarity determining region (CDR) of a first antibody having the second protein structure, and wherein the second portion of the first protein structure includes a second complementarity determining region (CDR) of a second antibody having the first protein structure.

21. A computer-implemented method, comprising:
identifying a protein sequence computation model and a protein structure computation model;
applying the protein sequence computation model to generate, based at least on an input protein sequence, a plurality of proposed protein sequences;
identifying, based at least on the plurality of proposed protein sequences, a set of possible amino acid residues for each position in at least a portion of an output protein sequence; and
generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.

22. A non-transitory computer readable medium storing instructions, which when executed by at least one data processor, result in operations comprising;
identifying a protein sequence computation model and a protein structure computation model;
applying the protein sequence computation model to generate, based at least on an input protein sequence, a plurality of proposed protein sequences;
identifying, based at least on the plurality of proposed protein sequences, a set of possible amino acid residues for each position in at least a portion of an output protein sequence; and
generating, using the protein structure computation model, a first protein structure having the output protein sequence by applying the protein structure computation model to select, for each position in at least the portion of the output protein sequence, a possible amino acid residue from the set of possible amino acid residues for inclusion in the output protein sequence.
